(19) United States
(12) Patent Application Publication
(10) Pub. No.: US 2023/0250490 A1
(43) Pub. Date: Aug. 10, 2023

(54) Microbially Induced Inflammation Produces Changes in Distinct Healthy Tissues in Human Oral Cavity

(71) Applicant: Colgate-Palmolive Company, New York, NY (US)

(72) Inventors: Dandan CHEN, Bridgewater, NJ (US); Harsj Mahendra TRIVEDI, Hillsborough, NJ (US); James MASTERS, Ringoes, NJ (US); Richard P. DARVEAU, Camano Island, WA (US); Jeffrey S. MCLEAN, Seattle, WA (US)

(73) Assignee: Colgate-Palmolive Company, New York, NY (US)

(21) Appl. No.: 18/167,664

(22) Filed: Feb. 10, 2023

Related U.S. Application Data
(60) Provisional application No. 63/308,543, filed on Feb. 10, 2022.

(51) Int. Cl. C12Q 1/689

(52) U.S. Cl. CPC C12Q 1/689; C12Q 2600/106

(57) ABSTRACT

Methods of identifying an individual as being a slow gingivitis responder or a high gingivitis responder are disclosed. The methods are based on the temporal difference in the shift of the ratio of Bacteroidetes to Firmicutes in subgingival plaque samples taken from healthy tissue that is distant from the site of plaque induced inflammation. Methods of treating gingivitis and methods of preventing, reducing the severity of or delaying onset of gingivitis in an individual who is a slow gingivitis responder or a high gingivitis responder are disclosed.

### REFERENCE TO AN ELECTRONIC SEQUENCE LISTING

The contents of the electronic sequence listing (Colgate 14220US Sequence Listing.xml; size: 330,284 bytes; and date of creation: Feb. 10, 2023) is herein incorporated by reference in its entirety.

### BACKGROUND

The gums, also referred to as gingiva, which are part of the soft tissue lining of the mouth, surround the teeth and provide a seal around them. The gingival margin is the interface between the sulcular epithelium and the epithelium of the oral cavity. This interface exists at the most coronal point of the gingiva, otherwise known as the crest of the marginal gingiva. The gingival crevice, also called gingival sulcus, is the space located around a tooth between the wall of the unattached gum tissue and the enamel and/or cementum of the tooth.

Healthy gums are firm and pale pink and fitted tightly around the teeth. Classic signs and symptoms of gingivitis include red, swollen, tender gums that may bleed when brushing or flossing. Oral infections are often correlated with systemic health issues.

Gingivitis is an inflammation of the gums that is the initial stage of gum disease. The direct cause of gingivitis is the unwanted microbial colonization by pathogenic bacteria on the teeth and gums. Pathogenic bacteria can for example produce toxins that can irritate the gum tissue, causing gingivitis. At this early stage in gum disease, damage can be reversed, since the bone and connective tissue that hold the teeth in place are not yet affected. Left untreated, however, gingivitis can become an advanced stage of gum disease, periodontitis, and cause permanent damage to teeth and jaw.

Periodontitis, a chronic and irreversible form of periodontal disease, is an age associated gingival inflammatory disease that is more prevalent than cardiovascular disease and affects more than 795 million adults globally. Periodontitis is associated with a dysbiotic dental plaque enriched in gram-negative bacteria like Porphyromonas and Tannerella species. If left untreated, periodontitis results in a dysregulated and chronic host immune response leading to irreversible structural tissue damage and bone loss. Periodontal disease has even been associated with other systemic diseases in humans, including arthritis, endocarditis, bacterial pneumonia, type-2 diabetes, and Alzheimer's. Gingivitis, a reversible and milder form of periodontal disease, is considered a precursor in the development for the majority of periodontitis cases. However, a direct connection between the etiopathogenesis leading from gingivitis to periodontitis remains unresolved. Additionally, gingivitis and periodontitis generally occur within localized tooth sites, though susceptible individuals may develop multiple diseased sites over different time periods and across their lifespan.

Gingivitis can be treated and resolved with good oral hygiene, such as longer and more frequent brushing, flossing and the use of an antiseptic mouthwash. The earlier gingivitis is treated the less chance of permanent damage. Accordingly, methods of diagnosing gingivitis during early stages of gingivitis allow for treatment to be initiated before the condition advances. Such methods can also be adapted to monitoring oral health and treatment over time.

A recent study, which utilized an experimental gingivitis model (EG), not only verified previously observed clinical variations in host response to plaque-induced inflammation reported in the literature (High and Low response), but also identified a novel clinical response type, Slow (Bamashmous et al., 2021 Human Variation in Gingival Inflammation. Proc Natl Acad Sci USA July 6; 118(27) e2012578118, which is incorporated herein by reference in its entirety). These variations in gingival inflammation observed in humans are now proposed as clinical Inflammatory Response Types (IRTs). Each of these phenotypes revealed distinctly defining clinical, microbial, and host mediator dynamics. In brief, High-IRT have a rapid plaque growth rate, rapid increase in gram-negative Bacteroidetes, high levels of host mediators, and high levels of inflammation; Low-IRT have a rapid plaque growth rate, rapid increase in gram-negative Bacteroidetes, low levels of host mediators, and low levels of clinical inflammation; and Slow-IRT have a delayed plaque growth rate, sustained higher levels of gram-positive Streptococci, and delayed onset of clinical inflammation yet ultimately reached high levels of inflammation—similar to High-IRT.

Despite the delayed onset of high levels of inflammation in Slow-IRT, both High-IRT and Slow-IRT each present as high levels of inflammation. After high levels of inflammation are reached, High-IRT and Slow-IRT can be distinguished from each other by measuring levels of IL-1β in a sample of gingival crevicular fluid from the individual at the site of the gingivitis (the gingivitis IL-1β level). The IL-1β level is significantly unchanged compared to the IL-1β level in gingival crevicular fluid measured pre-gingivitis (the baseline or pre-gingivitis IL-1β level) in an individual who is a Slow-IRT. By contrast, in an individual that is a High-IRT, the gingivitis IL-1β level is significantly elevated compared to the pre-gingivitis IL-1β level.

Understanding the defenses and physical reaction, such as the status of and changes to oral commensal bacteria population throughout the inflammatory process from initial development of plaque to gingivitis and periodontal disease allows for development and implementation of more effective preventative care and treatments, and products therefor. Determining whether an individual is a Slow-IRT or a High-IRT provides insight useful in provided effective treatment and preventive care for that individual. For individuals identified as Slow-IRTs, treatment and preventive care using oral care compositions comprising ingredients having antimicrobial activity and free of additional ingredients that have anti-inflammatory activity is preferred. For individuals identified as High-IRTs, treatment and preventive care using oral care compositions comprising ingredients having antimicrobial activity and ingredients having anti-inflammatory activity is preferred. (U.S. Provisional Application 63/200,002, which is incorporated herein by reference in its entirety).

### BRIEF SUMMARY

Methods of identifying an individual as being a slow gingivitis responder or a high gingivitis responder based on temporal variations in bacterial composition of the oral microbiome in heathy tissue at sites distant from the site of plaque induced inflammation are provided. The methods comprising the steps of: obtaining a pre-gingivitis-derived subgingival plaque sample from the individual when the individual has been identified as not having gingivitis. The bacterial composition of the pre-gingivitis-derived subgingival plaque sample is analyzed to determine the ratio of Bacteroidetes to Firmicutes present in the pre-gingivitis-derived subgingival plaque sample (i.e., the baseline iFBR). Two additional samples are obtained at different time points in the development of plaque induced inflammation associated with gingivitis and each additional sample analyzed. An early distant healthy-derived subgingival plaque sample is obtained from the individual and the bacterial composition of the early distant healthy-derived subgingival plaque sample is analyzed to determine the ratio of Bacteroidetes to Firmicutes present in the early distant healthy-derived subgingival plaque sample (i.e., the early iFBR). A late distant healthy-derived subgingival plaque sample is obtained from the individual and the bacterial composition of the late distant healthy-derived subgingival plaque sample is analyzed to determine the ratio of Bacteroidetes to Firmicutes present in the late distant healthy-derived subgingival plaque sample (i.e., the late iFBR). The baseline iFBR, the early iFBR and the late iFBR are compared. If the early iFBR is significantly unchanged compared to the baseline iFBR and the late iFBR is significantly changed compared to the baseline iFBR, the individual is identified as a slow gingivitis responder. If the early iFBR is significantly changed compared to the baseline iFBR and the late iFBR is significantly changed compared to the baseline iFBR, the individual is identified as a high gingivitis responder.

In some embodiments, such methods of identifying an individual as being a slow gingivitis responder or a high gingivitis responder further comprise the steps of examining the individual and determining that the individual does not have gingivitis prior to obtaining pre-gingivitis-derived subgingival plaque sample from the individual, and examining the individual and determining that the individual has plaque induced inflammation associated with gingivitis prior to obtaining an early distant healthy-derived subgingival plaque sample from the individual and prior to obtaining a late distant healthy-derived subgingival plaque sample from the individual.

Methods of treating an individual who has been identified as having gingivitis are provided. Such methods may eliminate, ameliorate or delay or prevent progression of symptoms and disease. The methods comprise identifying the individual as being a slow gingivitis responder or a high gingivitis responder. If the individual is identified as a slow gingivitis responder, one or more oral care compositions comprising one or more ingredients having antimicrobial activity and free of additional ingredients that have anti-inflammatory activity are applied to the individual's oral cavity. If the individual is identified as a high gingivitis responder, one or more oral care compositions comprising one or more ingredients having antimicrobial activity and one or more ingredients having anti-inflammatory activity are applied to the individual's oral cavity.

Methods of preventing gingivitis and periodontal disease in an individual who has been identified as not having gingivitis are provided. Such methods may prevent, reduce the severity of or delay onset of symptoms and disease. The methods comprise identifying the individual as being a slow gingivitis responder or a high gingivitis responder. If the individual is identified as a slow gingivitis responder, one or more oral care compositions comprising one or more ingredients having antimicrobial activity and free of additional ingredients that have anti-inflammatory activity are applied to the individual's oral cavity. If the individual is identified as a high gingivitis responder, one or more oral care compositions comprising one or more ingredients having antimicrobial activity and one or more ingredients having anti-inflammatory activity are applied to the individual's oral cavity.

In some embodiments of the methods of treating and preventing gingivitis, the one or more ingredients having antimicrobial activity that are used in such methods of is/are selected from the group consisting of: arginine, zinc phosphate, zinc oxide, zinc citrate, triclosan, digluconate, thymol, menthol, eucalyptol, methyl salicylate, saline, antibiotics and fluoride.

In some embodiments of the methods of treating and preventing gingivitis, the one or more ingredients having anti-inflammatory activity that are used in such methods is/are selected from the group consisting of: chlorhexidine, DHA and vitamin D.

In some embodiments of the methods of treating and preventing gingivitis, the one or more oral care compositions used in such methods is/are selected from the group consisting of: a tooth paste, an oral rinse and a mouthwash.

### BRIEF DESCRIPTION OF THE DRAWINGS

FIGS. 1A-1J contain data showing that control sites are not static and vary by Inflammatory Responder Type. FIG. 1A shows Plaque Index (PI) stratified by Inflammatory Responder Type (IRT) over the induction phase (Day 0-21) with respective Controls. FIG. 1B shows linear regression of mean PI by IRT Test and Control over the induction phase. FIG. 1C shows Gingival Index (GI) stratified by IRT over the induction phase with respective Controls. Red dashed line represents a GI value of 1.5 which represents significant clinical inflammation within gingiva tissues. FIG. 1D shows linear regression of mean GI by IRT Test and Control over the induction phase. FIG. 1E shows Bleeding on Probing (BOP) stratified by IRT over the induction phase with respective Controls. FIG. 1F shows linear regression of mean BOP by IRT Test and Control over the induction phase. FIG. 1G shows Gingival crevicular fluid (GCF) volume stratified by IRT over the induction phase with respective Controls. FIG. 1H shows linear regression of mean GCF volume by IRT Test and Control over the induction phase. FIG. 1I shows Bacterial Load (16S copies) stratified by IRT over the induction phase with respective Controls. FIG. 1J shows linear regression of mean Bacterial Load by IRT Test and Control over the induction phase. Boxes represents data and medians±interquartile ranges; whiskers and outliers>1.5 IQR below (above) the 25th (75th) percentile. Trend lines represent loess regression mean values across all time points. Statistical analysis was performed using the non-parametric Wilcoxon-Rank Sum Test adjusted by FDR. Significance level indicated by asterisks. Significance levels: ns=non-significant, *P<0.05, **P<0.01, and ***P<0.001.

FIGS. 2A-2D contain data showing changes in subgingival plaque diversity were observed in Healthy Control Sites and varied by Inflammatory Responder Type. FIG. 2A shows Alpha diversity measured by Observed Amplicon Sequence Variants (ASVs) and Shannon Indices. Statistical Significance calculated by Wilcoxon Rank Sum Test Adjusted by FDR. FIG. 2B shows Bray-Curtis Dissimilarity Index for each Inflammatory Responder Type (IRT) Test compared to Day 0 over the Induction phase (Day 0-21). FIG. 2C shows Bray-Curtis Dissimilarity Index for each IRT Controls compared to Day 0 over the Induction phase (Day 0-21). FIG. 2D shows Beta Diversity calculated using Weighted Unifrac Distances and visualized by Principal Coordinate Analysis for each IRT with Test and Controls. Marginal Boxplots indicate PC1 and PC2 over the Induction phase (Day 0-21). Boxes represent data and medians±interquartile ranges; whiskers and outliers>1.5 IQR below (above) the 25th (75th) percentile. Trend lines represent loess regression mean values across all time points. Dashed lines represent test sites and solid lines represent control sites. Statistical analysis was performed using the non-parametric Wilcoxon-Rank Sum Test adjusted by FDR. Significance level indicated by asterisks. Significance levels: ns=non-significant, *P<0.05, **P≤0.01, and ***P≤0.001.

FIGS. 3A-3E show microbiome compositions shift within healthy control sites in a Responder Dependent Manner. FIG. 3A shows the Percent relative abundance of agglomerated count data at the phylum level for the 6 most abundant phyla. Dashed lines represent test sites and solid lines represent control sites by clinical Inflammatory Responder Type (IRT). FIG. 3B shows Z-scored heatmaps of relative abundance using agglomerated data at the genus level. Heatmaps were then organized and grouped based off gram stain designation including Gram-positive, Gram-negative, as well as by members of the Candidate Phyla Radiation (CPR). Key bacteria from each gram classification are highlighted in red to compare across different responder test and control sites. Most are members of the Firmicutes and Bacteroidetes phyla or are associated with periodontal disease. FIGS. 3C-3E show Percent relative abundance of agglomerated data at the phylum level grouped by IRT test and control sites over the induction period. Trend lines represent mean values across all time points. Whiskers represent standard error.

FIGS. 4A and 4B show the Inverse Firmicutes/Bacteroidetes Ratio (iFBR) by Inflammatory Responder Type. The inverse Firmicutes/Bacteroidetes Ratio (iFBR) was generated using relative abundance data agglomerated to the phylum level. The dashed red line represents 0 on the Log 2 scale as a reference across plots. FIG. 4A represents the iFBR for test and control sites among the different clinical Inflammatory Responder Types (IRTs) with their respective controls. FIG. 4B represents the iFBR for test and control sites stratified by IRT. The dashed red line represents 0 on the Log 2 scale as a reference across plots. Boxes represent data and medians±interquartile ranges; whiskers and outliers>1.5 IQR below (above) the 25th (75th) percentile. Trend lines represent loess regression mean values across all time points. Dashed lines represent test sites and solid lines represent control sites. Statistical analysis was performed using the non-parametric Wilcoxon-Rank Sum Test adjusted by FDR. Significance level indicated by asterisks. Significance levels: ns=non-significant, *P<0.05, **P≤0.01, and ***P≤0.001.

FIGS. 5A-5D show Amplicon Sequence Variants are detected contralaterally between Test and Control sites. ASV level data was converted to a presence-absence matrix and plotted as a heatmap in order to identify contralateral detection between test and control sites over the induction period (Day 0-21) by clinical Inflammatory Responder Type (TRT). Gram negative and Candidate Phyla Radiation (CPR) genera that were enriched in test sites were selected for this analysis resulting in a total of 3,509 ASVs. Red text (Darker) represents ASVs that were detected simultaneously between test and control sites by subject. Blue text (Lighter) represents ASVs that were detected in test sites prior to any detection in control sites by subject. FIG. 5A shows High-IRT by subject resulted in 29 (0.83%) ASVs that were contralaterally detected. 6/6 subjects had a contralaterally detected ASV. FIG. 5B shows Low-IRT by subject resulted in 15 (0.43%) ASVs that were contralaterally detected. 3/6 subjects had a contralaterally detected ASV. FIG. 5C shows Slow-IRT by subject resulted in 89 (2.5%) ASVs that were contralaterally detected. 8/9 subjects had a contralaterally detected ASV. FIG. 5D is a Table of the Gram negative and CPR genera used in this analysis, the number of species detected within those genera as well as the number of corresponding ASVs for each genera.

FIGS. 6A-6L show maturing plaque in Test Sites induces Host Mediator changes in Control Sites which precede a shift in the Control Site Microbiome. FIGS. 6A-6C show Z-scored heatmap of log fold change of chemokines compared to baseline (Day 0) among different clinical Inflammatory Responder Type (IRT) control sites. Key inflammatory mediators are highlighted in red text (lighter). FIGS. 6D-6F show IL-8 (Left y-axis), IL-6, and TNF-a (Right y-axis) among IRT control sites. Red box (shaded) highlights shift in host mediators in control site. FIGS. 6G-6I show Percent Relative Abundance of Firmicutes (Left y-axis) and Bacteroidetes (Right y-axis) using agglomerated data at the phylum level. Trendlines represent the mean value. Solid lines represent control sites and dashed lines represent test sites. Labels represent TRT test and control sites by Phylum (i.e., HTF—High Test Firmicutes, HCF—High Control Firmicutes, HTB—High Test Bacteroidetes, HCB—High Control Bacteroidetes). Red boxes (shaded) represent the dysbiotic shift in control sites. FIGS. 6J-6L show a graphical interpretation of the temporal relationships of the microbiome and host mediators between test and control sites over the induction phase (Day 0-21).

### DETAILED DESCRIPTION

The terms “slow gingivitis responder”, “slow responder” and “Slow-IRT” are used interchangeably.

The terms “high gingivitis responder”, “high responder” and “High-IRT” are used interchangeably.

The terms “low gingivitis responder”, “low responder” and “Low-IRT” are used interchangeably.

The terms “non-gingivitis-derived subgingival plaque sample” and “pre-gingivitis-derived subgingival plaque sample” are used interchangeably and refer to a subgingival plaque sample obtained from the individual when the individual has been identified as not having gingivitis. Pre-gingivitis-derived subgingival plaque sample provide a sample to identify and determine baseline levels of constituents of the subgingival microbiome and the determination of relative abundance of such constituents.

The terms “an early distant healthy-derived” obtained 21-28 days after a professional teeth cleaning or 7-14 days after the appearance of localized plaque-induced inflammation from a site of healthy tissue that is distant from the site of gingivitis, i.e., the site of localized plaque-induced inflammation, in an individual that has localized plaque-induced inflammation. An “early contralateral subgingival plaque sample” is an example of an early distant healthy-derived subgingival plaque sample.

An “early distant healthy-derived sample subgingival plaque sample refers to a subgingival plaque sample obtained from a site of healthy tissue that is distant from the site of gingivitis, i.e., the site of localized plaque-induced inflammation, in an individual that has localized plaque-induced inflammation, 7-14 days after the appearance of localized plaque-induced inflammation, or if an induction procedure based on the experimental gingivitis model is employed (which includes a 14-day pre-induction period that begins with a cleaning and examination a day followed by a 21-day induction period during which time there is a cessation of brushing), subgingival plaque sample is obtained 21-28 days after the start of the pre-induction phase, which is 7-14 days after the start of the induction phase. An “early contralateral subgingival plaque sample” is an example of a late distant healthy-derived subgingival plaque sample.

A “late distant healthy-derived sample subgingival plaque sample refers to a subgingival plaque sample obtained from a site of healthy tissue that is distant from the site of gingivitis, i.e., the site of localized plaque-induced inflammation, in an individual that has localized plaque-induced inflammation, after 14 days after the appearance of localized plaque-induced inflammation, or if an induction procedure based on the experimental gingivitis model is employed (which includes a 14-day pre-induction period that begins with a cleaning and examination a day followed by a 21-day induction period during which time there is a cessation of brushing), subgingival plaque sample is obtained after 28 days after the start of the pre-induction phase, which is 7-14 days after the start of the induction phase. A “late contralateral subgingival plaque sample” is an example of a late distant healthy-derived subgingival plaque sample.

The term “inverse Firmicutes/Bacteroidetes ratio (iFBR)” refers to the ratio of Bacteroidetes to Firmicutes present in a subgingival plaque sample.

The terms “pre-gingivitis iFBR”, “non-gingivitis iFBR” and “baseline iFBR” are used interchangeably and refer to the iFBR in a pre-gingivitis-derived subgingival plaque sample.

The terms “early iFBR” is used to refer to the iFBR in an early distant healthy-derived subgingival plaque sample.

The terms “late iFBR” is used to refer to the iFBR in a late distant healthy-derived subgingival plaque sample.

“Statistically elevated” and “significantly elevated” are used interchangeably and refer to one amount being higher than another and the difference being statistically significant.

“Unchanged” and “significantly unchanged” are used interchangeably and refer to one amount being the same or nearly the same as another and the difference not being statistically significant.

“Oral care composition” refers to a composition that is delivered to the oral surfaces. The composition may be a product which, during the normal course of usage, is not, the purpose of systemic administration of particular therapeutic agents, intentionally swallowed, but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, and the like.

The stability in host-microbial interface is essential for health across mucosal surfaces in the human body. Barrier immunity is not characterized by the absence of bacteria but by their regulated presence under healthy immune surveillance. This has also been termed the para-inflammatory state and is required for tissues to respond to insult and restore homeostasis. This is especially relevant on mucosal surfaces where there is a constant microbial challenge to the host immune system. For example, in oral mucosal surfaces, one of the main protective mechanisms of tissue and therefore, host protection from unwanted microbial colonization is the constant highly orchestrated transit of neutrophils from the local periodontal vasculature through healthy gingival tissue and into the gingival crevice. There, neutrophil surveillance is essential for maintaining the proper amount and composition of dental plaque, a highly evolved and organized bacterial consortium found on the tooth surface that actively contributes to normal periodontal tissue function.

Oral commensal bacteria actively participate with gingival tissue to maintain healthy neutrophil surveillance and normal tissue and bone turnover processes. Disruption of this homeostatic bacterial—host relationship occurs during experimental gingivitis studies where it has been clearly established that increases in the number and type of oral commensal bacteria increase clinical indices of inflammation. Studies in germ-free mice have revealed that dental plaque is essential for proper neutrophil homing and also contributes to normal alveolar bone turnover processes. Proper neutrophil monitoring of the dental plaque microbial biofilm therefore results in a process termed “healthy homeostasis” with the consequence being both colonization resistance, a microbial protection mechanism which resists infection as well as maintaining the appropriate microbial composition for normal periodontal bone and tissue function.

Model systems for studying host-microbe interaction dynamics during inflammatory events directly in humans in a well-controlled and reversible way are limited. A unique advantage of studies in the oral cavity is the availability of one such model, experimental gingivitis (EG). Deliberate cessation of oral hygiene allows for the high-resolution study of the initiation and development of gingival inflammation, as well as understanding the variation in the severity which exists across the human population. This model permits the development and maturation of normal dental plaque, a rich biofilm, along the gingival margin and within the periodontal pocket surrounding human teeth which results in a relatively rapid host response as inflammation progresses to a clinically diseased state. Furthermore, this model also allows for the investigation of the effects of localized inflammation in distant otherwise healthy tissues in the human oral cavity.

Accumulation of dental plaque in the human induced gingivitis experimental model is a convenient and reproducible model facilitating the study of the disruption of healthy tissue homeostasis. The human experimental gingivitis model offers the unique advantage of monitoring disease development in real time in order to study the change from a model have revealed rapid alterations in clinical measures of inflammation that parallel microbial plaque biomass increases and compositional changes during the development of gingivitis. Furthermore, it has been reported that in human experimental gingivitis studies the subject-based susceptibility to plaque-induced gingival inflammation is an individual trait. It has been reported that individual responses to induced gingivitis could be grouped into high and low clinical phenotypes with the high response phenotype being linked to a persistent hyper-responsive para-inflammatory state. Although nearly every human gingivitis study since 1965 has recognized there is variation in clinical parameters to bacterial dental plaque accumulation, the factors responsible for the significantly different individual host responses have not been elucidated. In this report, three different clinical response groups were identified and a granular parallel analysis of these groups revealed unique host and microbiome characteristics during induced inflammation.

Periodontal disease, including periodontitis and gingivitis, is a localized inflammatory disease of the periodontium which is characterized by a progressive destruction of the tissues supporting the tooth. However, the primary etiology in which healthy teeth become susceptible or how this disease spreads beyond localized sites of infection in humans remains less clear. Being able to identify biomarkers, such as key bacteria, associated with the transition from health to disease may provide additional insight into the mechanisms by which healthy tooth sites become susceptible to disease and/or disease progression. Furthermore, understanding relationships associated with the different variations observed in the gingival inflammatory response, represented by an individual's clinical Inflammatory Responder Type (TRT), may indicate an individual's risk of getting periodontal disease and/or progressing to the more devastating and irreversible forms.

Initially, two IRTs were identified, a High IRT, in which host experiences extensive plaque induced inflammation, and a Low IRT in which the host does not experience as severe an inflammatory response to plaque. The recent discovery of a Slow IRT in which host experiences extensive plaque induced inflammation but such reaction is delayed has provided new insights for maintaining good oral health and informs the treatment most effective to combat and prevent plaque induced inflammation and gingivitis.

Provided herein is a new method of distinguishing High-IRTs from Slow-IRTs. The temporal changes within healthy control teeth during a recent experimental gingivitis study are described herein. Microbially-induced inflammation was produced in test sites for a period of 21 Days in a well-controlled and reversible way within young generally healthy adults. By analyzing the subgingival microbiome using subgingival plaque, significant changes in the subgingival microbiome community have been identified. Importantly, these changes were followed by similar trends observed within distant test sites located contralaterally in the mouth. That is, the subgingival microbiome undergoes changes in healthy tissue at sites distant from the site of plaque induced inflammation. Methods are provided which entail analyzing sample from healthy tissue at sites distant from the site of plaque induced inflammation and treating an individual based upon the results of such analysis.

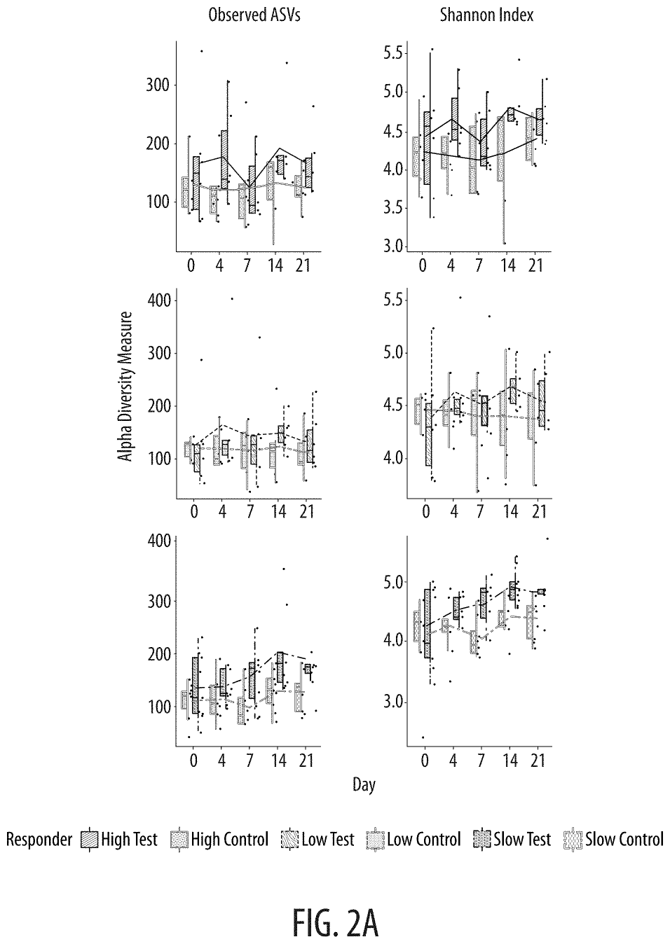

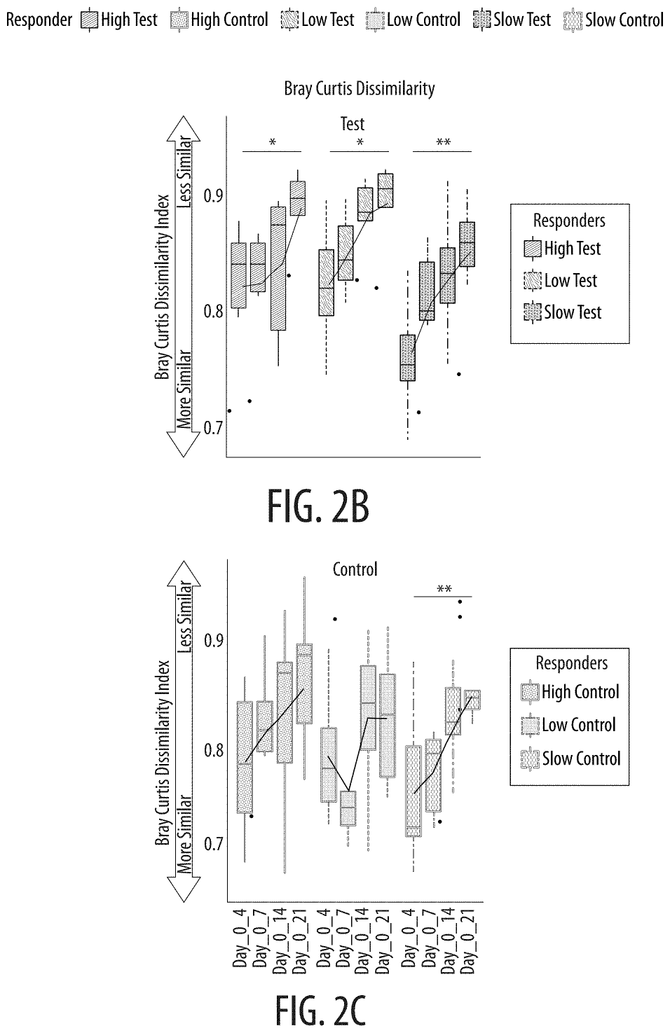

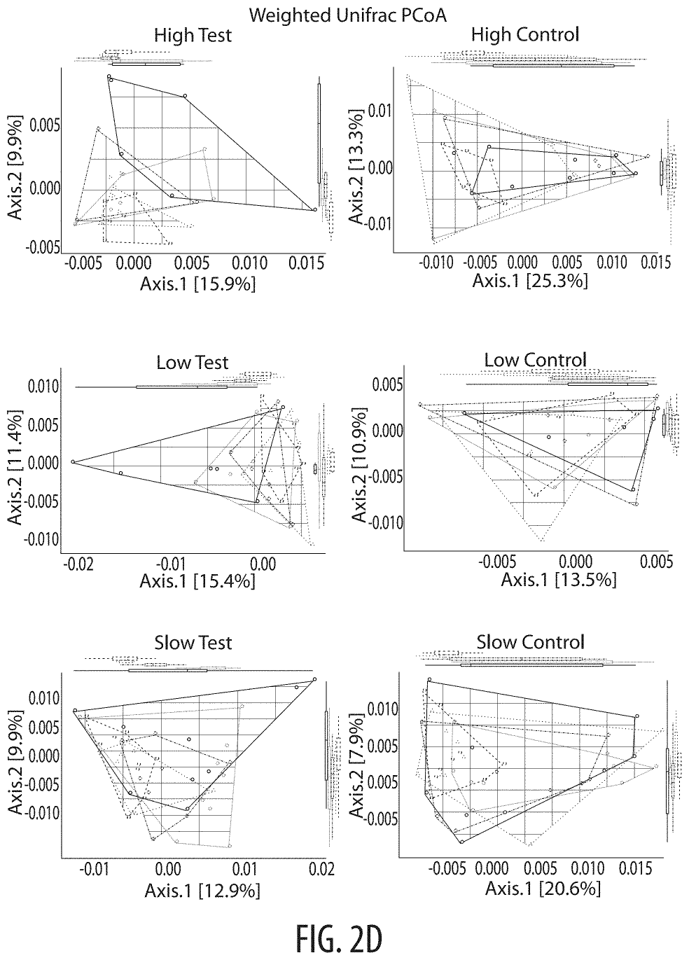

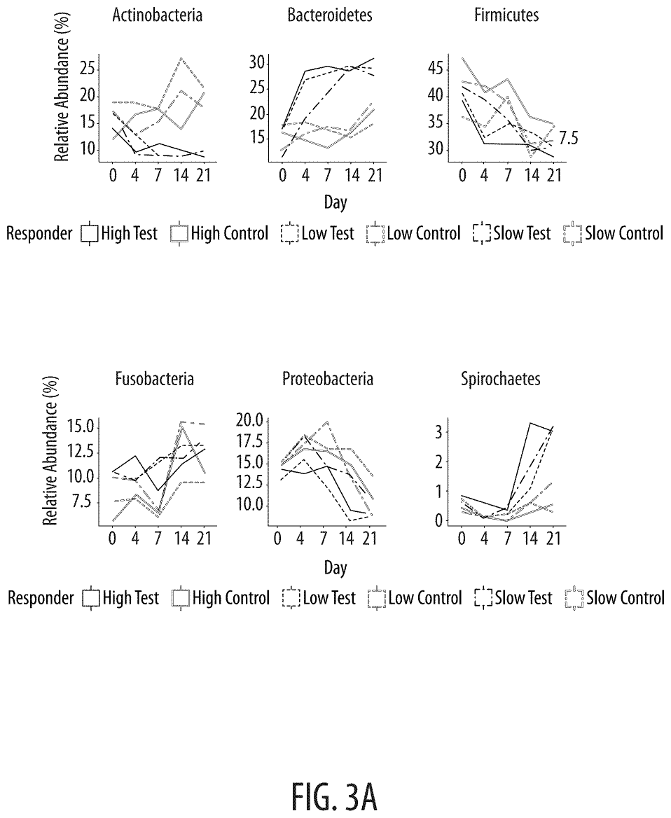

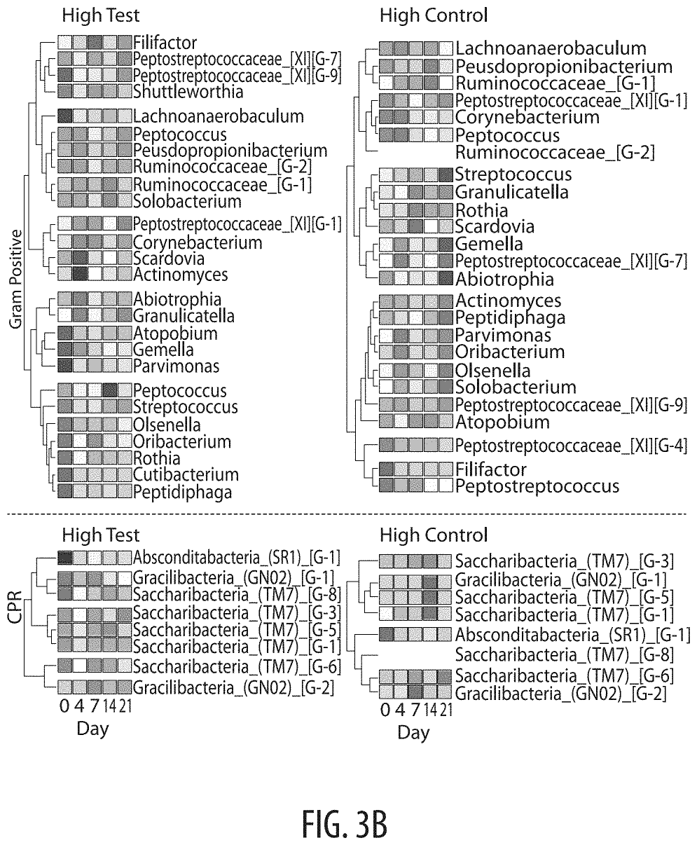

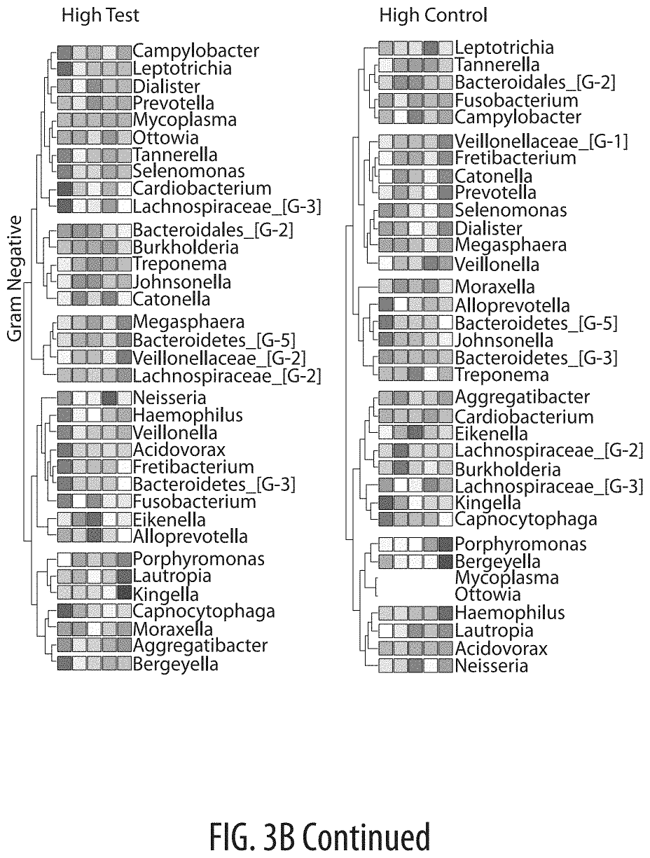

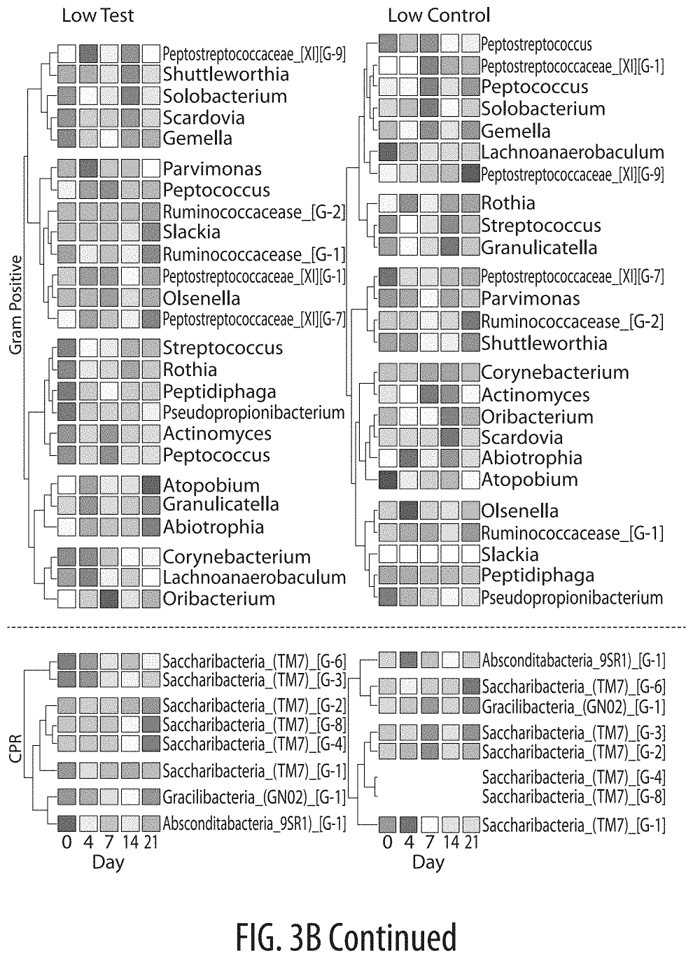

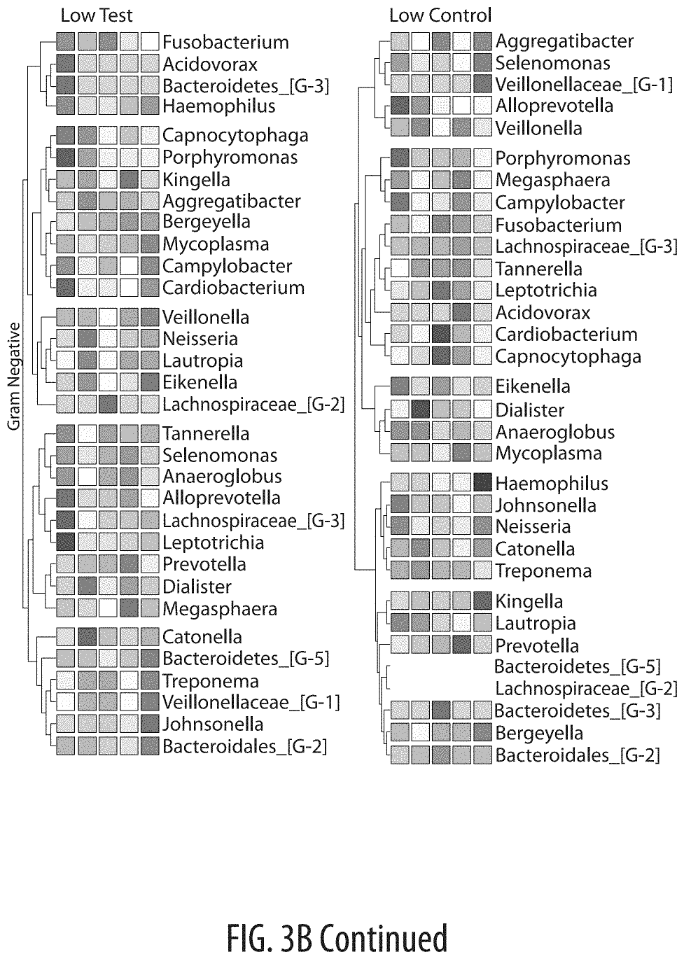

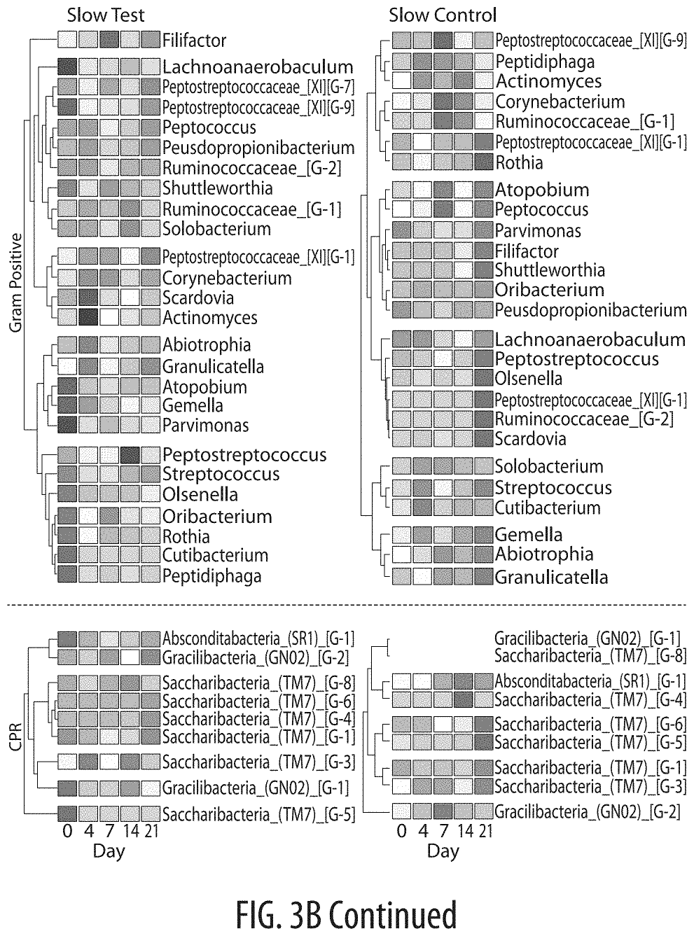

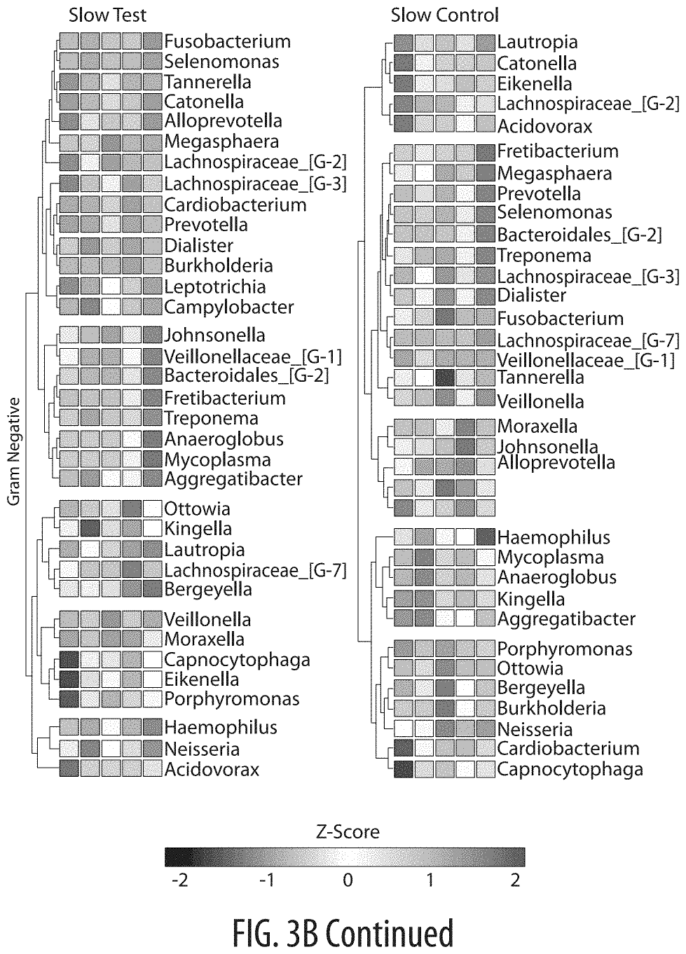

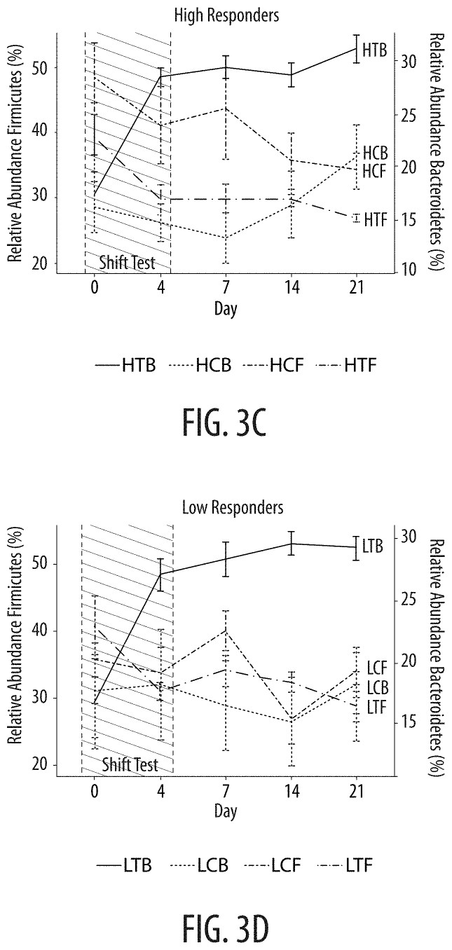

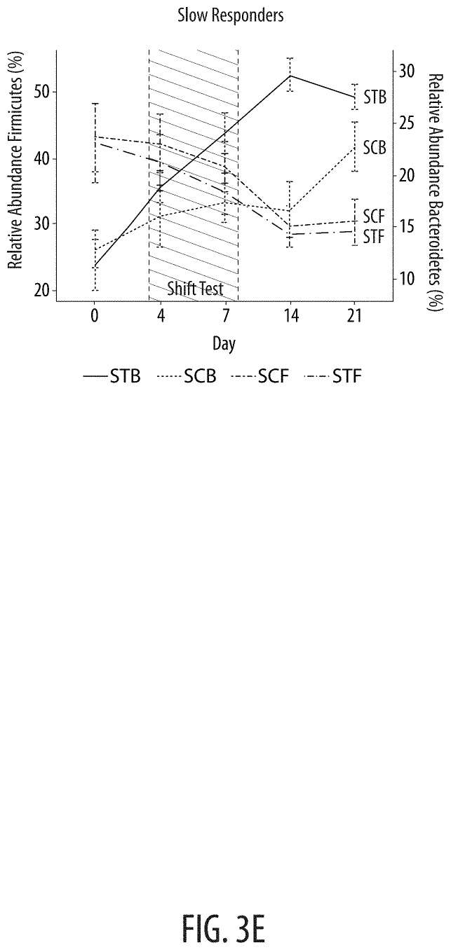

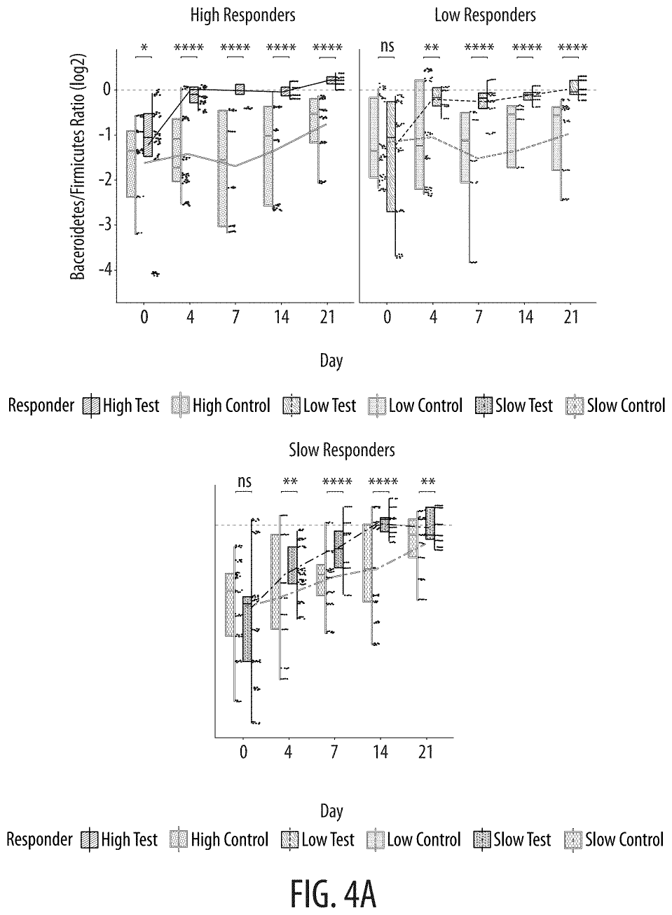

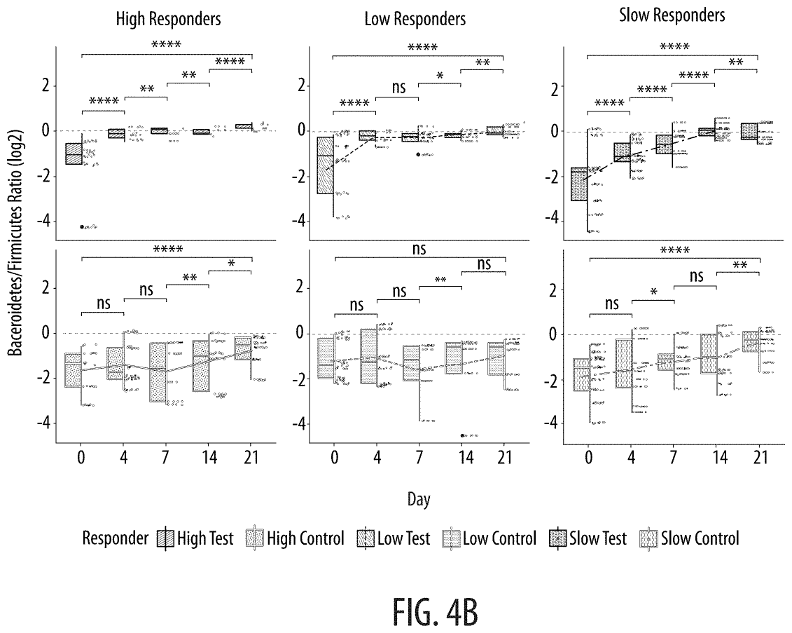

Taxonomic diversity from healthy control teeth changed over the 21 Day period of induced inflammation. Slight increases were seen in alpha diversity in High- and Slow-IRT groups while remaining rather stable in the Low-IRT (FIG. 2A). Furthermore, similar shifts in beta diversity and Bray-Curtis dissimilarity measures were observed between all IRT test and control sites (FIGS. 2B-2D). Taxonomic analysis revealed that a majority of the subgingival microbiome community detected in this study was made up of two major phyla, Firmicutes and Bacteroidetes (FIGS. 3A and 3C). In oral health, Firmicutes are generally associated with Gram positive commensal bacteria while Bacteroidetes are generally associated with Gram negative dysbiotic bacteria. This is corroborated by a genus level analysis which looked at the relative abundance of different genera stratified by Gram stain designations within test and control sites for all IRTs (FIG. 3B). However, in human gut microbiome research, the opposite trend is observed with Bacteroidetes being associated with gut health and commensalism while Firmicutes are associated with dysbiosis and disease. Additionally, in human gut microbiome research the use of a Firmicutes/Bacteroidetes ratio (FBR) has been accepted as an index for measuring the state of the gut microbiome. The inverse Firmicutes/Bacteroidetes ratio (iFBR) may be used as an index for assessing subgingival microbiome health/dysbiosis (FIGS. 4A and 4B). Application of the proposed iFBR stratified by IRT test and control sites revealed a temporal relationship between the iFBR and onset of significant clinical inflammation within High- and Slow-IRT test sites. This trend was also observed in the High- and Slow-IRT control sites, although delayed (FIGS. 4A and 4B). Interestingly, even though the Low-IRT was observed to shift towards a Bacteroidetes dominant community in test sites as early as Day 4, similar to the High-IRT, the iFBR remained rather stable in distant control sites over the Induction phase (Day 0-21) unlike the other IRTs.

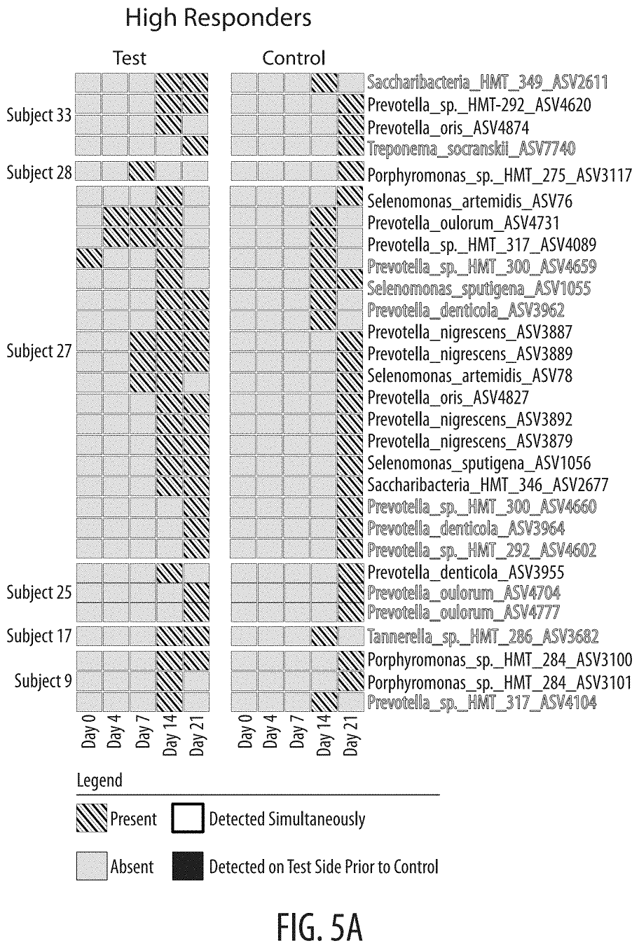

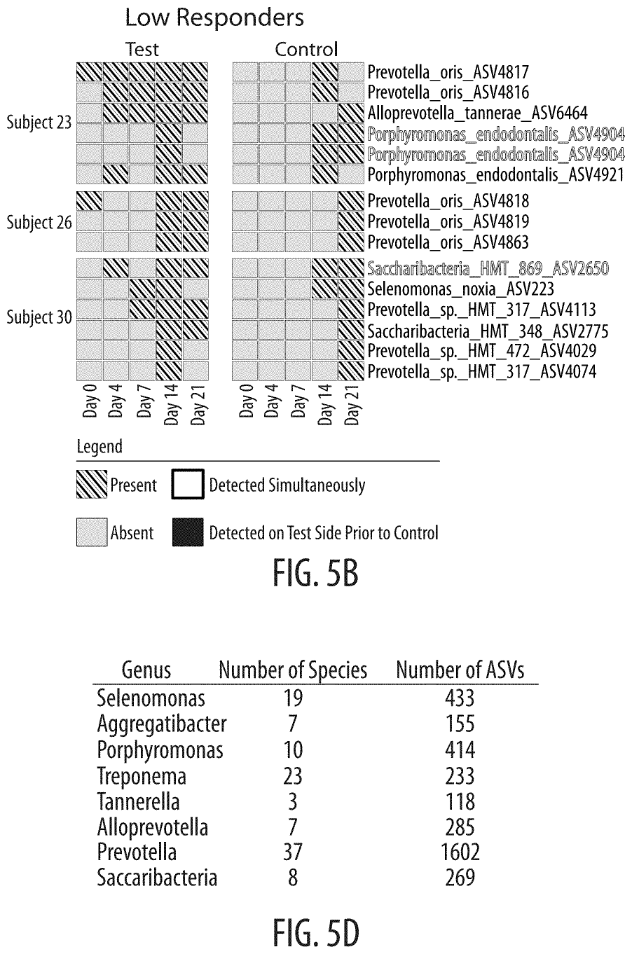

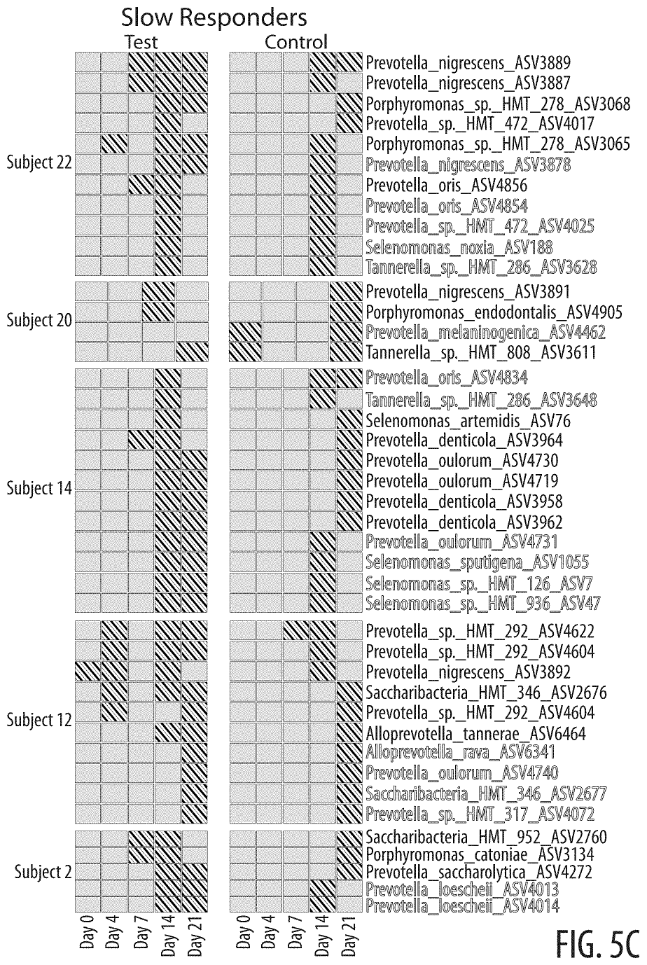

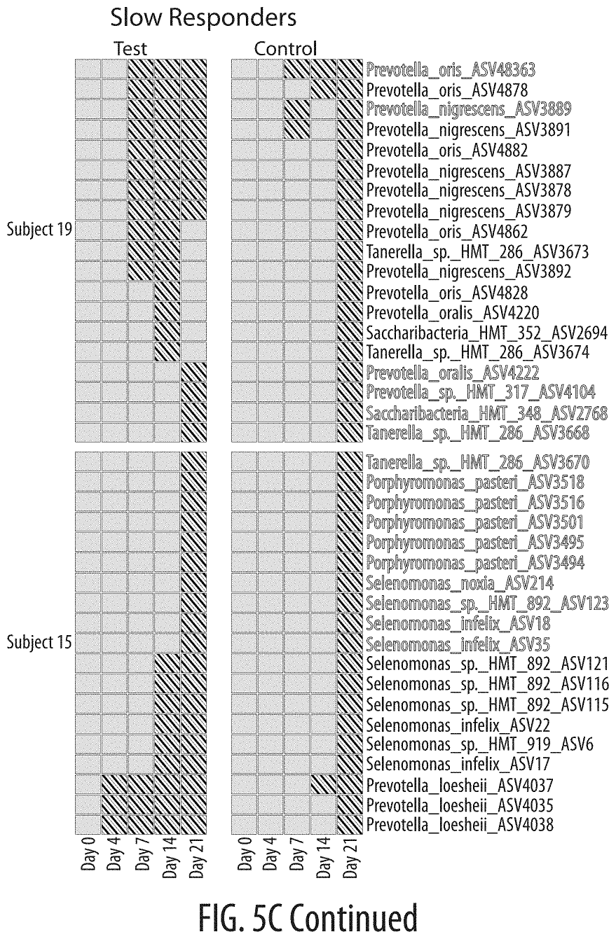

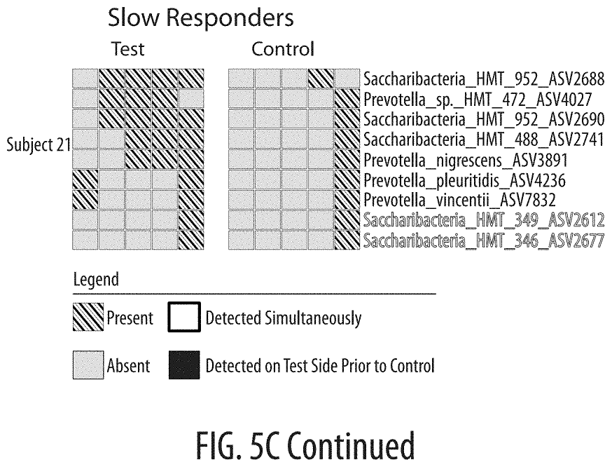

Taxonomic data at the strain level was also examined. A high-resolution method to investigate amplicon sequence variants (ASVs), which represent strain level diversity in 16S amplicon data, was applied in order to identify if a particular strain could be driving these observations within and or across individuals. Due to the large variation in relative abundance between individuals, day, and sample sites, a presence/absence approach to ASVs within the Bacteroidetes genera in addition to the Saccharibacteria was applied due to the strong associations with oral inflammation. These taxa are summarized in FIG. 5D. Briefly, the analysis revealed high variability within and between individuals, yet a number of ASVs detected in test sites prior to control sites or simultaneously within test and control sites during the Induction phase were identified (FIGS. 5A-5D). Interestingly, many of these ASVs were detected near the end of the induction phase between test and control sites. The analysis supports the notion that as plaque matures on teeth, eventually resulting in dispersion, it is possible to detect the same ASV in distant sites in the mouth. This is a possible mechanism by which other sites might become susceptible to periodontal disease or colonization of periodontal pathogens.

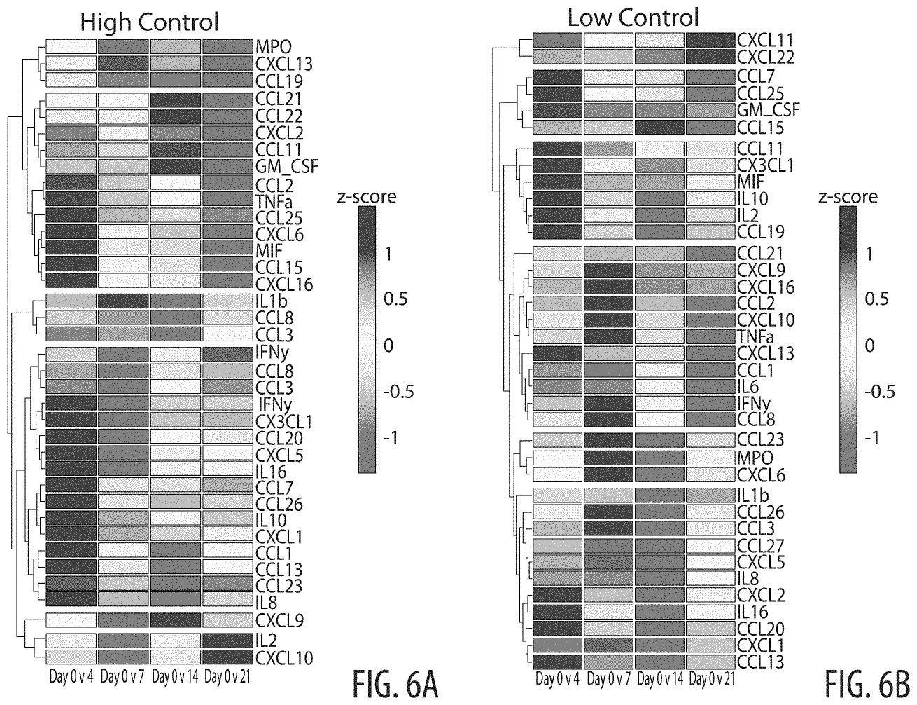

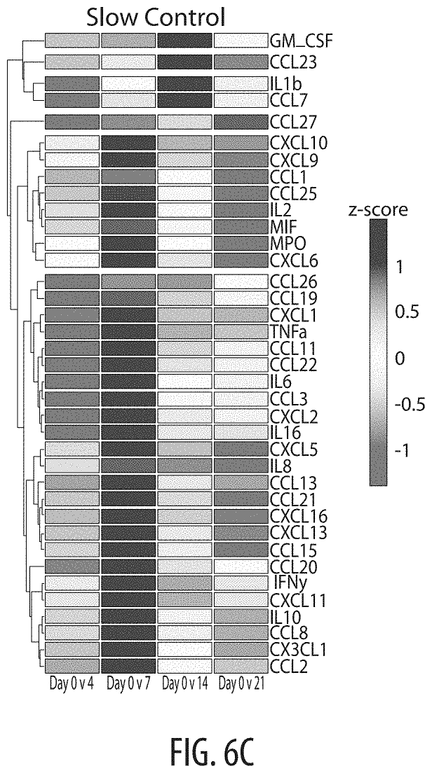

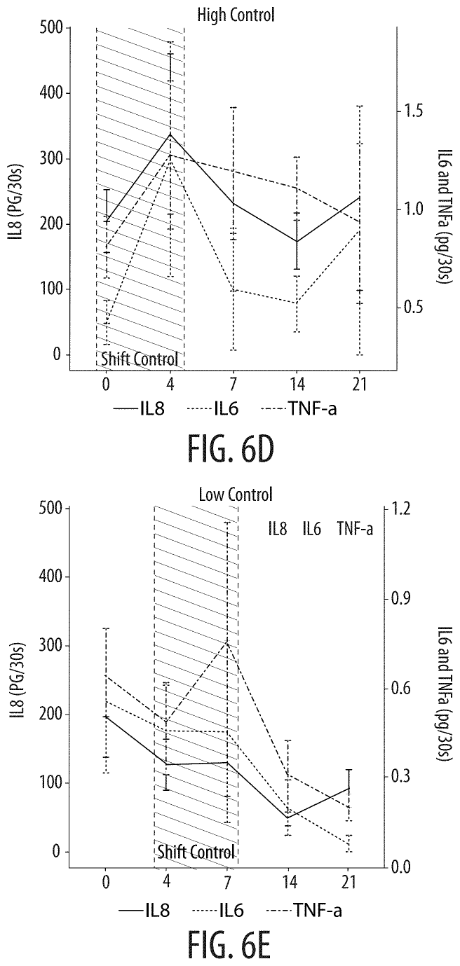

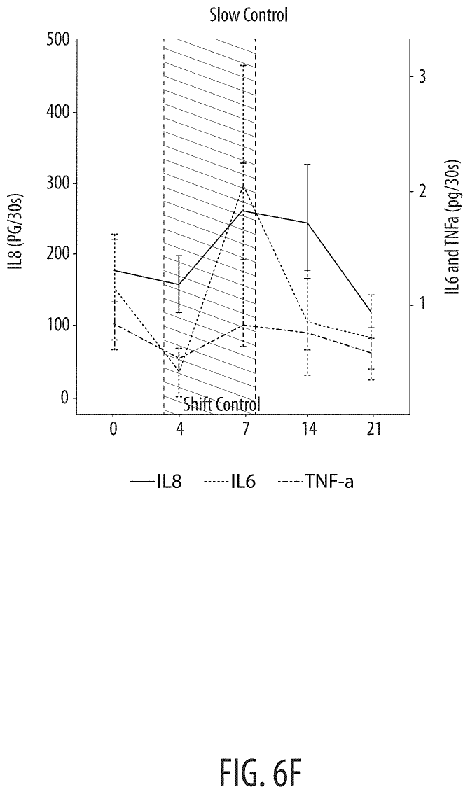

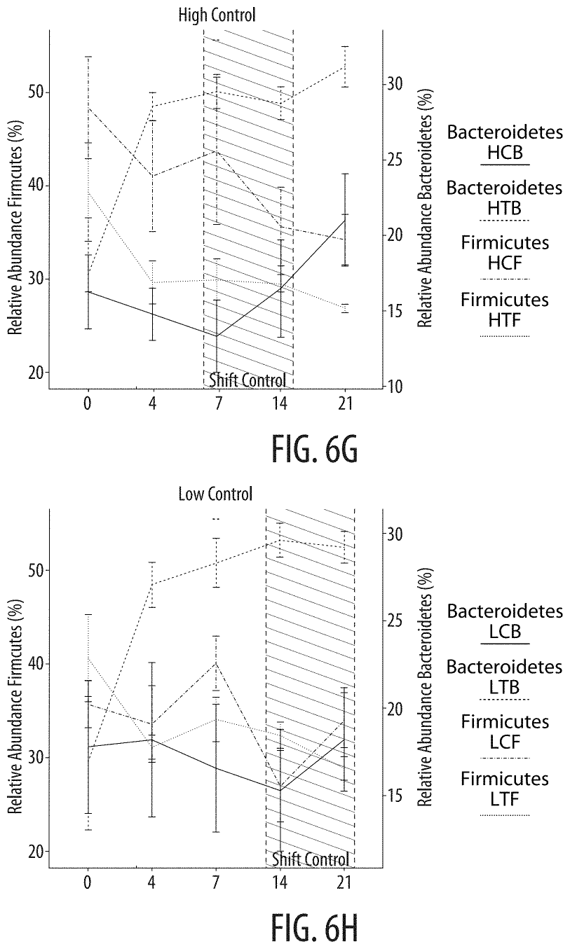

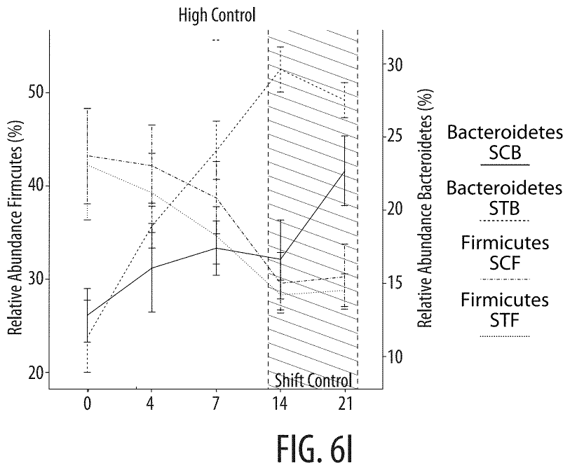

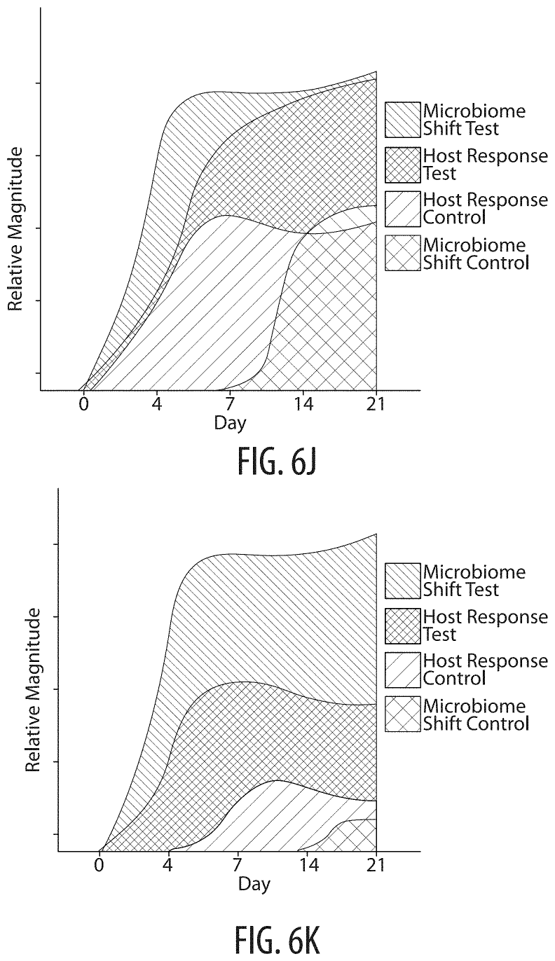

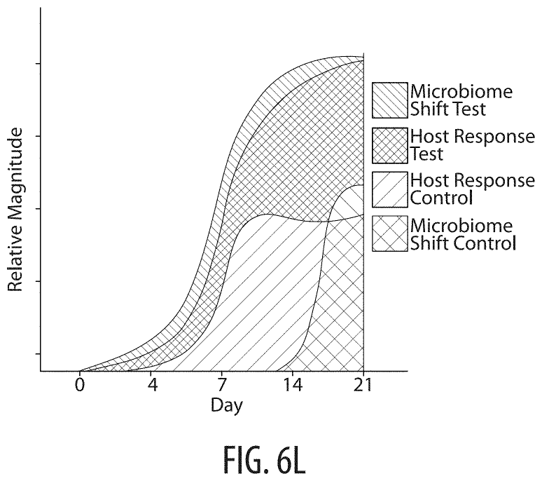

The temporal analysis of subgingival microbiome data, specifically the relative abundance of the two most abundant phyla Firmicutes and Bacteroidetes, supports the conclusion that normal plaque accumulation and maturation occurring within test sites results in a change in community composition towards dysbiosis. This suggests that subclinical changes in host mediators when coupled with a maturing biofilm elsewhere in the mouth, may facilitate a subgingival environment more favorable for Bacteroidetes members and may provide a mechanism in which localized periodontal inflammation can cause distant healthy tooth sites to become susceptible. Furthermore, the analysis revealed that this relationship varies by clinical Inflammatory Responder Type (IRT) and indicates that different IRTs may have different levels of risk associated with periodontal disease and/or disease progression. An illustrative interpretation of these temporal dynamics is provided for each IRT (FIGS. 6J-6K).

The changes observed in healthy control sites is highly correlated with changes we observed within respective test sites. Due to the fact that these changes seem to be TRT dependent as well as show variation between test and control sites, it is not likely that these are normal colonization processes after a professional cleaning. The Low-TRT, which are able to modulate their immune response and/or maintain periodontal homeostasis despite significant plaque accumulation and community maturation, seem to have a lower propensity to affect distant healthy sites within the mouth. This may suggest that members of the Low-IRT have a lower risk of healthy tooth sites becoming infected and Low-TRT is a more clinically desirable phenotype in comparison to the High- and Slow-IRT. Low-IRT produces lower levels of inflammation than the High- and Slow-IRTs despite a similarly rapid plaque accumulation as High-IRT and shift towards a dysbiotic community composition as seen in High- and Slow-IRT

According to methods provided herein, pre-gingivitis-derived subgingival plaque samples are obtained and used to determine a baseline iFBR. Subsequently, an early distant healthy-derived subgingival plaque sample and a late distant healthy-derived subgingival plaque sample are obtained and used to determine the early iFBR and late iFBR, respectively. If the early iFBR has changed significantly from the baseline iFBR, the individual is identified as a High-IRT and can be treated accordingly. If the early iFBR has not changed significantly from the baseline iFBR, but the late iFBR has changed significantly from the baseline iFBR, the individual is identified as a Slow-IRT and can be treated accordingly.

Methods of collecting subgingival plaque samples are well known to those skilled in the art. Pre-gingivitis-derived subgingival plaque samples may be collected when the individual is experiencing no localized plaque-induced inflammation. An early distant healthy-derived subgingival plaque sample is obtained 7-14 days after the appearance of localized plaque-induced inflammation from a site of healthy tissue, not from the site of inflammation. A late distant healthy-derived subgingival plaque sample is obtained after 14 days after the appearance of localized plaque-induced inflammation from a site of healthy tissue, not from the site of inflammation. In some embodiments, the plaque induced inflammation associated with gingivitis may be intentionally induced as part of a procedure to determine the inflammatory response type of an individual. After a cleaning and examination to confirm the absence of gingivitis, pre-gingivitis-derived subgingival plaque samples may be taken and used to establish a baseline iFBR. In some embodiments, baseline iFBR is determined using a sample collected on day 0. The cleaning and examination start a 14-day hygiene phase pre-induction period (day −14 to day 0). On day 0, the individual begins a 21-day induction period in which the individual refrains from brushing the entire mouth or a side of the mouth as in the experimental gingivitis model during which time the plaque induced inflammation associated with gingivitis develops. Subgingival plaque samples may be taken at one or more sites of healthy tissue distant from a site of where inflammation has developed on days 7-14 of the induction phase (7-14 days after cessation from brushing; 21-28 days after cleaning and baseline sample collection; early distant healthy-derived subgingival plaque samples), such as healthy tissue sites contralateral to a site of inflammation, to be analyzed and establish early iFBR. Subgingival plaque samples may be taken at one or more sites of healthy tissue distant from a site of where inflammation has developed after 14 days of the induction phase (after 14 days after cessation from brushing; after 28 days after cleaning and baseline sample collection; late distant healthy-derived subgingival plaque samples), such as healthy tissue sites contralateral to a site of inflammation to be analyzed and establish late iFBR.

Methods of collecting subgingival plaque samples are well known. In some embodiments, sterile paper points (STER-I-CELL Paper Points, Size M; Coltene, Whaledent, Cuyahoga Falls, Ohio, USA) are inserted into the gingival sulcus of the six maxillary teeth for 30 seconds to collect samples. Multiple samples can be taken and pooled. DNA can be extracted using a commercially available kit (e.g., QIAamp DNA Microbiome Kit; Qiagen, Germany).

DNA extraction methods may include positive and negative controls. Quantitative real-time PCR is performed to determine the total bacterial load in each sequenced sample. A qPCR standard curve may be generated using known genomic DNA.

Analysis of merged reads is performed using the Quantitative Insights into Microbial Ecology QIEVIE2 following the Divisive Amplicon Denoising Algorithm 2 (DADA2) pipeline workflow to generate amplicon sequence variants (ASV's). Taxonomic assignment to classify ASV's can be performed using the Human Oral Microbiome Database (HOMD 16S rRNA RefSeq v. 15.1).

Sample purification and determination of DNA concentrations can be performed using commercially available purification kits to further purify and increase the DNA yield. Determination of DNA concentrations can also be performed using commercially available kits.

Comprehensive microbial profiling of subgingival plaque samples can be performed via high throughput sequencing of 16S rRNA gene following the standard Illumina Miseq System protocol. Briefly, amplification of DNA is performed using primers with overhang Illumina flow cell adapter sequences targeting hypervariable (V3 and V4) regions of the bacterial 16 s rRNA gene.

Samples can be amplified using commercially available PCR kits Amplicons can be purified using magnetic beads and indexed and the indexed PCR amplicons can be further purified with magnetic beads. A commercially available normalization kit can be used for library normalization followed by sequencing.

Analysis of sequencing data can be performed using automation. Taxonomic assignment to classify the amplicon sequence variants (ASV's) can be performed using a database that contains predominately full length 16S rRNA gene reference sequences.

Community richness is measured by observed species: total count of unique ASV's in the sample and by the nonparametric richness estimator Chao1, which accounts for the number of singletons and doubletons. Total community diversity (richness and evenness) may be measured by Simpson's inverse diversity index, Shannon index, and Faith's phylogenetic diversity (PD), which uses phylogenetic distances to calculate alpha diversity.

Beta diversity, between samples diversity, was determined using phylogenetic-based Unifrac distances, which measure phylogenetic distance between samples for both unweighted (presence\absence), weighted (relative abundance), and ASV based Bray-Curtis dissimilarity matrices that accounts for both the presence/absence and abundance of unique ASV's. Beta diversity metrics were calculated with ordinate function in “phyloseq” and were visualized by non-metric multidimensional scaling (NMDS) plots using plot oridination function in “phyloseq”. Quantitative real-time PCR can be performed to determine the total bacterial load in each sequenced sample.

In some embodiments, constituents present in the microbiome can be identified and their relative abundance determined by routing methods. In some embodiments, genomic DNA is extracted from the subgingival plaque using a QIAamp DNA Mini Kit (QIAGEN Sciences, USA). In some embodiments, an extra lysozyme treatment (3 mg/ml, 1.5 h) for bacterial cell lysis is performed. DNA concentration and purity are measured such as with a NanoDrop ND-1000 spectrophotometer (Thermo Fisher Scientific, USA) and monitored on 1% agarose gels. 16S rRNA Gene Sequencing is used to analyze the microbiome. The 16S rRNA V4 gene may be used to evaluate the bacterial composition and diversity using Illumina Hiseq (Novogene Bioinformatics Technology Co., Ltd.). Polymerase chain reaction (PCR) amplification of the V4 region of the bacterial 16S rRNA gene may be performed using specific primers such as 515F and 806R. Sequencing libraries may be generated using TruSeq™ DNA PCR-Free Sample Preparation Kit (Illumina, USA) following manufacturer's recommendations The library may be sequenced on an Illumina HiSeq 2,500 and 250 bp paired-end reads were generated and the raw reads may be deposited into the NCBI Sequence Read Archive (SRA) database.

In some embodiments, methods are provided to of treating an individual who has been identified as having gingivitis. Methods of treatment may comprise identifying that the individual as being a slow responder or a high responder and then treating the individual based upon whether they are a slow responder or a high responder.

Individuals identified as having gingivitis may be treated to resolve the gingivitis based upon whether they are a slow responder or a high responder. Methods of treating an individual who has gingivitis comprise identifying an individual who has gingivitis as being a slow responder or a high responder by a method described above and then treating such individual based upon whether the individual is identified as a slow responder or a high responder.

In some embodiments, the individual who has gingivitis and has been identified as being a slow responder may be treated by applying to the individual's oral cavity an oral care composition that is an anti-bacterial oral rinse. In some embodiments, the individual who has gingivitis and has been identified as being a slow responder may be treated by applying to the individual's oral cavity an oral care composition comprising one or more ingredients selected from the group consisting of: arginine, zinc phosphate, zinc oxide, zinc citrate, triclosan, digluconate, thymol, menthol, eucalyptol, methyl salicylate, saline, antibiotics and fluoride. In some embodiments, the individual does not gingivitis but has been identified as being a slow responder. The slow responder may be treated prophylactically by applying to the individual's oral cavity an oral care composition comprising one or more ingredients selected from the group consisting of arginine, zinc phosphate, zinc oxide, zinc citrate, triclosan, digluconate, thymol, menthol, eucalyptol, methyl salicylate, saline, antibiotics and fluoride.

In some embodiments, the individual who has gingivitis and has been identified as being a high responder may be treated by applying to the individual's oral cavity an oral care composition comprising anti-bacterial components such as one or more ingredients selected from the group consisting of arginine, zinc phosphate, zinc oxide, zinc citrate, triclosan, digluconate, thymol, menthol, eucalyptol, methyl salicylate, saline, antibiotics and fluoride and further comprising anti-inflammatory components such as one or more ingredients selected from the group consisting of chlorhexidine, DHA and vitamin D. In each instance, the various components may be included in a single oral care composition or in two or more separate oral care compositions. In some embodiments, the individual does not gingivitis but has been identified as being a high responder. The high responder may be treated prophylactically by applying to the individual's oral cavity an oral care composition comprising Di gluconate anti-bacterial components such as one or more ingredients selected from the group consisting of arginine, zinc phosphate, zinc oxide, zinc citrate, triclosan, digluconate, thymol, menthol, eucalyptol, methyl salicylate, saline, antibiotics and fluoride and further comprising anti-inflammatory components such as one or more ingredients selected from the group consisting of chlorhexidine, DHA and vitamin D.

### EXAMPLE

A study was designed with the aim to characterize changes observed in distant healthy control sites located contralaterally on the maxilla in relation to gingival inflammation being induced in test sites from normal plaque accumulation and maturation over a period of 21 Days. Significant alterations within the human oral cavity that directly results from induced inflammation occurring in distant sites within the oral cavity were observed. Notably, increases in pro-inflammatory host mediators associated with periodontal inflammation, IL-8, IL-6, IL1-b, and TNF-a, as well as a dysbiotic ecological shift in the subgingival microbiome, represented by the proposed inverse Firmicutes/Bacteroidetes ratio (iFBR), within healthy controls without any significant clinically observed inflammation within these distant healthy sites. This effect was influenced by an individual's Inflammatory Responder Type (IRT). Microbial shifts in healthy sites were most evident in the High- and Slow-IRT with the High-IRT having a rapid contralateral effect and the Slow-IRT having a delayed contralateral effect. Interestingly, the Low-IRT, which are able to modulate their immune response within test sites and do not obtain the same elevated levels of inflammation as the High- and Slow-IRT, have a muted contralateral effect. These findings bolster our understanding of the variation in specific host inflammatory response types to localized plaque-induced inflammation and the subsequent effects within distant healthy tissues that impacts both the host mediator profiles and microbial community composition. Such changes may represent precursor events leading to increased risk of gingival inflammation in distant otherwise healthy sites in the human oral cavity.

### Methods

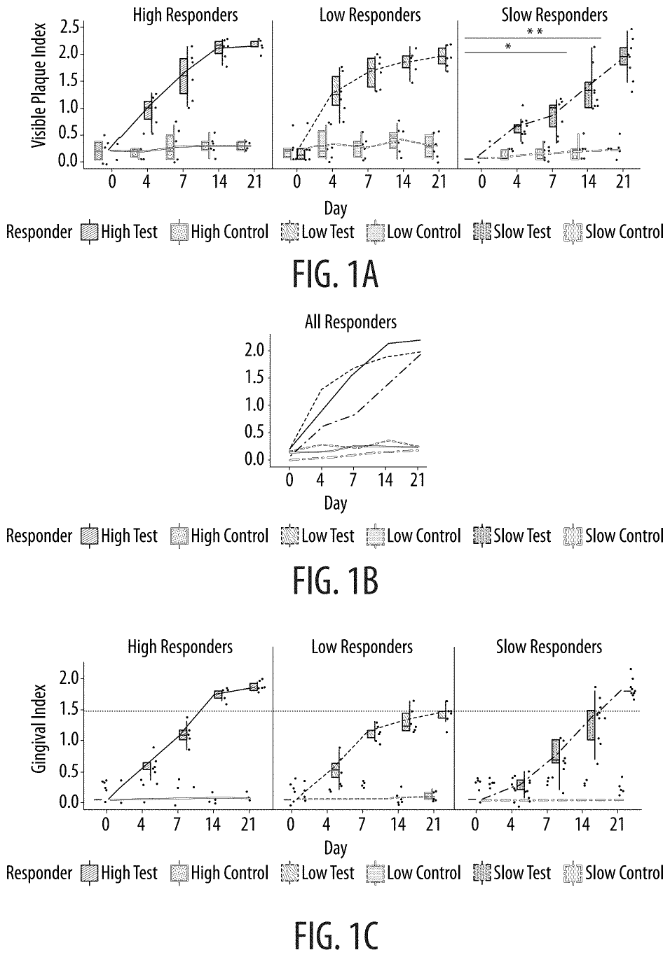

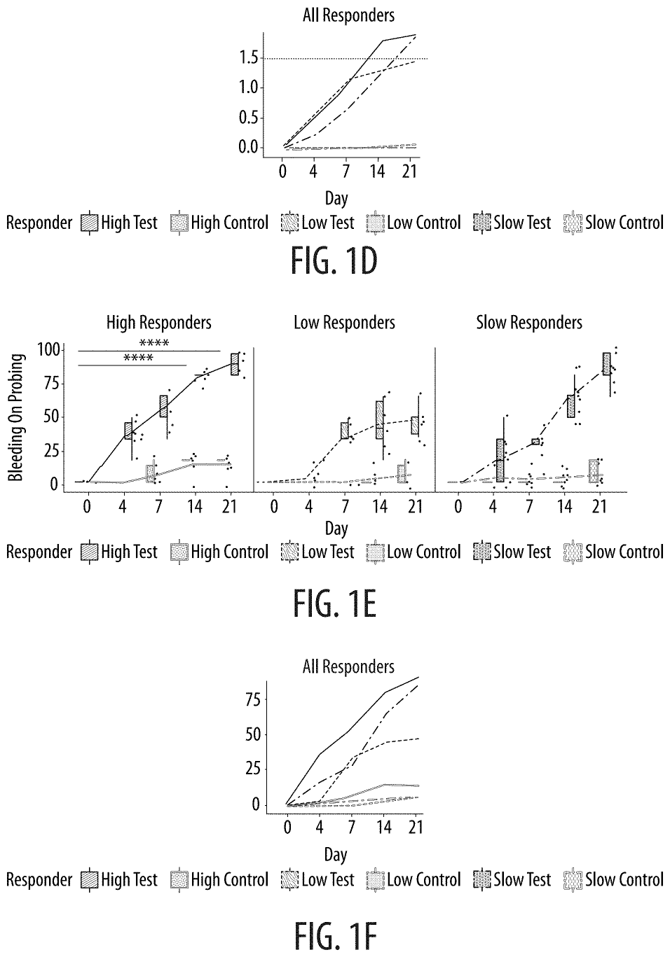

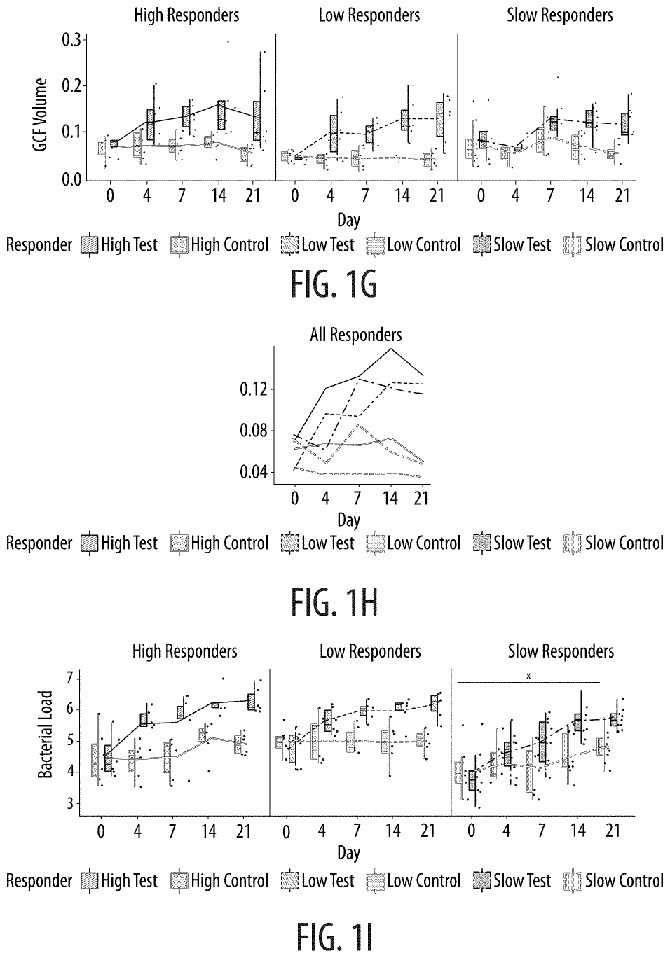

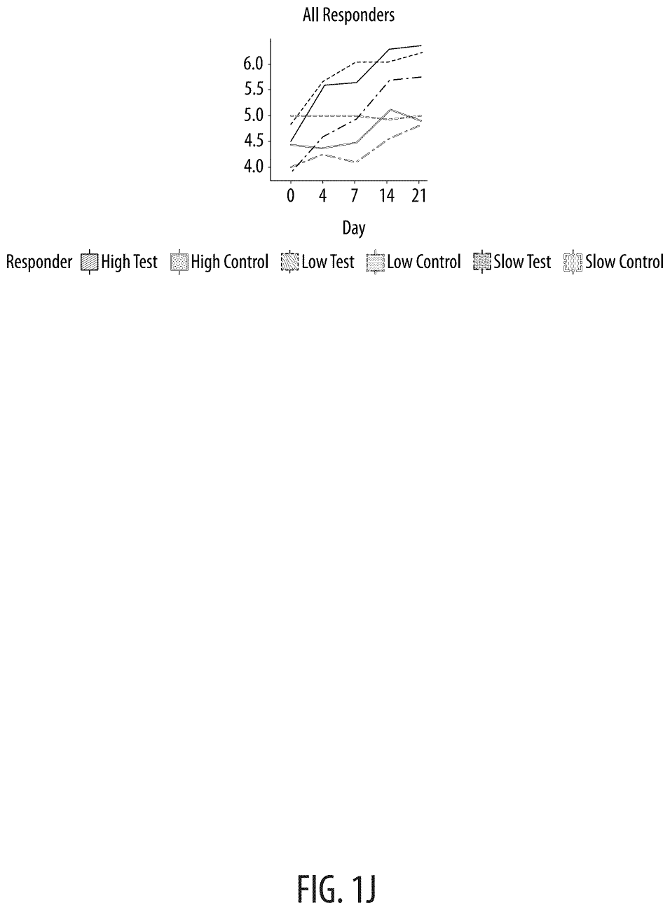

The study included the following phases 1) Hygiene phase for two weeks prior to baseline (Day −14-Day 0), 2) Gingivitis induction phase lasting for three weeks (Day 0-Day 21), and 3) Resolution phase for two weeks (Day 21-Day 35) (FIG. 1A). During the experimental induction phase the subjects were given customized intraoral stents that prevented oral hygiene at the experimental sites. Fidelity monitoring of the intervention was conducted at each timepoint throughout the experiment by clinical assessment of the plaque index.

At Day −14, after obtaining informed consents and verification of inclusion/exclusion criteria, full clinical assessment and biospecimens collection were performed. After obtaining a maxillary impression for stent fabrication, full mouth prophylaxis was administered and thorough oral hygiene instructions were given. Participants returned 7 to 14 days after this initial visit and their baseline (Day 0) measurements and biospecimens collection were acquired. The acrylic stent was given to the participant with detailed instructions for use during regular brushing with the purpose of preventing accidental brushing of the experimental sites. Participants were instructed not to brush teeth on the test side (under the provided stent) and not to use any other measure of oral hygiene such as flossing or interdental aids. For the control side and rest of the mouth, participants were instructed to use the provided toothbrush (Colgate® Gum Comfort Toothbrush), toothpaste (Colgate® Cavity Protection Great Regular Flavor Fluoride Toothpaste), and dental floss (Oral B Glide), and to refrain from using mouth rinses and chewing gums during entire study period. Participants returned on Day 4 and on weekly basis afterward, clinical assessments were performed and samples collected each time using the same criteria as at the baseline visit. The course of the “no brushing” part of the experiment lasted 21 days to allow all the participants to develop gingivitis. After acquiring samples and clinical assessment on day 21, additional thorough prophylaxis was administered. Participants were given again detailed instructions in oral hygiene methods using the provided electric toothbrush (Philips Sonicare Electric Toothbrush), toothpaste (Colgate® Cavity Protection Great Regular Flavor Fluoride Toothpaste), and dental floss (Oral B Glide) beginning the same day and continued twice daily during the resolution phase. Assessment of gingival condition and biospecimens collection continued weekly during the reversal phase. Medical history and exclusion criteria were reviewed at each study visit.

The stent was fabricated to include only the occlusal surface of the study teeth and eliminate contact with the cervical margin of each tooth, thereby reducing the risk of plaque being disturbed during insertion or removal of the stent. The stent was constructed from 3-mm-thick plastic mouthguard material. Elimination of cervical contact was accomplished by blocking out around the gingival margin and proximal surfaces using a spacer made from 1-mm-thick mouthguard material. The stent was trimmed vertically on the buccal side to a length just short of the vestibule and extending 4-5 mm on the palatal side. Also, it was trimmed mesially to the middle of the canine, and distally to the middle of the second molar.

Subgingival plaque samples were collected at each study visit from both control and test sides. Sterile paper points (STER-I-CELL Paper Points, Size M; Coltene, Whaledent, Cuyahoga Falls, Ohio, USA) were inserted into the gingival sulcus of the six maxillary teeth for 30 seconds. At each study visit, a total of six samples per study side were collected and pooled and samples were transported to the lab on ice and then frozen at −80° C. until further analysis. DNA was extracted using a commercially available kit (QIAamp DNA Microbiome Kit; Qiagen, Germany) following the manufacturer's protocol, that uses both mechanical and chemical cell lysis. Sample purification and quality control were performed as previously described.

For DNA extraction negative controls were implemented by performing the DNA extraction protocol without plaque samples with either kit reagents only or kit reagents with the sterile paper points to assess for contamination. Also, to validate the efficiency of the technique, positive controls using known bacterial cultures were included. Quantitative real-time PCR was performed to determine the total bacterial load in each sequenced sample. A qPCR standard curve was generated from serially diluted Fusobacterium nucleatum ATCC 10953 genomic DNA.

Analysis of merged 300 bp paired-end reads (average length 450 bp) was performed using the Quantitative Insights into Microbial Ecology QIIME2 following the Divisive Amplicon Denoising Algorithm 2 (DADA2) pipeline workflow to generate amplicon sequence variants (ASV's). Taxonomic assignment to classify ASV's was performed using the Human Oral Microbiome Database (HOMD 16S rRNA RefSeq v. 15.1). Data were integrated into a single object using the “phyloseq” R package and further analyzed.

Sample purification was performed using a purification kit (the DNA Clean & Concentrator −5 kit; Zymo Research, Orange, Calif., USA) to further purify and increase the DNA yield. After DNA extraction and purification, DNA concentrations in the samples were determined fluorometrically (Quant-iT dsDNA HS Assay Kit; Invitrogen, Carlsbad, Calif., USA) with Fluorometer (Qubit 2.0; Life Technologies, Carlsbad, Calif., USA). Samples were stored at −20° C. until ready for sequencing.

Comprehensive microbial profiling of subgingival plaque samples was performed via high throughput sequencing of 16S rRNA gene following the standard Illumina Miseq System protocol. Briefly, amplification of DNA was performed using primers with overhang Illumina flow cell adapter sequences targeting hypervariable (V3 and V4) regions of the bacterial 16 s rRNA gene. The primers used were as follows:

SEQ ID NO: 1 was the 16S amplicon PCR
forward primer:
5′-TCGTCGGCAGCGTCAGATGTGTATAAGAGACAGCCTACGGGNG
GCWGCAG-3′
SEQ ID NO: 2 was the 16S amplicon PCR
reverse primer:
5′-TCTCGTGGGCTCGGAGATGTGTATAAGAGACAGGACTACHVGG
GTATCTAATCC-3′).

Samples were amplified in singletons in a 96 well plate format. Each reaction was performed using a PCR kit (KAPA HiFi HotStart ReadyMix; KAPA Biosystems, Boston, Mass., USA) in a total volume of 25 μl which included the following reagents: 2.5 μl of extracted DNA, 5 μl of both forward and reverse primers (1 μM each primer) and 12.5 μl of 2×KAPA HiFi HotStart ReadyMix. Amplicon PCR was performed on a thermocycler (C1000 Touch thermal cycler; BioRad, Hercules, Calif., USA) utilizing the following program: a denaturation stage at 95° C. for 3 minutes, followed by 35-40 cycles of denaturation at 95° C. for 30 seconds, annealing at 55° C. for 30 seconds and extension at 72° C. for 30 seconds, and then a final extension stage at 72° C. for 5 minutes. The generated amplicons from the first PCR were approximately 460 bp in size which was verified visually by running each reaction on 1% agarose gel electrophoresis at 100V for 30 minutes. Amplicons were subsequently purified using magnetic beads (Agencourt AMPure XP beads; Agencourt Bioscience Corporation, Beckman Coulter Inc., Beverly, Mass., USA) and indexed (Nextera XT v2 Index Kits, Set A, B, and D; Illumina, San Diego, Calif., USA). The indexing PCR conditions included a denaturation stage at 95° C. for 3 min, followed by 8 cycles of denaturation at 95° C. for 30 s, annealing at 55° C. for 30 s, and extension at 72° C. for 30 s, and then a final extension stage at 72° C. for 5 min. The indexed PCR amplicons were further purified with magnetic beads (Agencourt AMPure XP beads; Agencourt Bioscience Corporation, Beckman Coulter Inc., Beverly, Mass., USA), and the quality and size of the library were checked (High Sensitivity D1000 Reagents and Agilent 4200 TapeStation system; Agilent Technologies, Santa Clara, Calif., USA). Subsequently, library normalization was achieved using a normalization kit (SequalPrep Normalization Plate Kit; ThermoFisher Scientific, Waltham, Mass., USA). The normalized library was pooled and denaturated with sodium hydroxide (NaOH) (Fisher Scientific, Pittsburgh, Pa., USA). The denatured library (20 pM) was spiked with at least 20% control DNA (PhiX Control v3 library, Illumina, San Diego, Calif., USA) prior to loading to the sequencer. Paired-end sequencing was carried out on a sequencing platform (MiSeq System, Illumina, San Diego, Calif., USA) using a 2×300 cycle sequencing kit (MiSeq Reagent Kits v3, Illumina, San Diego, Calif., USA).

Analysis of sequencing data was performed using the Quantitative Insights into Microbial Ecology QIIME2 following the Divisive Amplicon Denoising Algorithm 2 (DADA2) pipeline workflow. A total of 16M paired-end reads were generated, average reads per sample was 41,017. Raw paired-end reads were imported to QIIME2 (v. 2018.2), following demultiplexing with Casava (v. 1.8), both forward and reverse reads were trimmed by 10 bp and truncated to 290 and 200 bp respectively. After quality filtering and denoising of sequences and removal of chimeras and singletons, a total of 2,219,156 high quality merged reads with a mean length of 452 bp were used for downstream analysis. Taxonomic assignment to classify the amplicon sequence variants (ASV's) was performed using the most up to date Human Oral Microbiome Database (HOMD 16S rRNA RefSeq v. 15.1), which is highly curated database that is specific for the human oral cavity and contains predominately full length 16S rRNA gene reference sequences. Samples were then filtered for taxonomic contaminants based on negative controls (kit reagents only or kit reagents with the sterile paper points). A phylogenetic tree was constructed using FastTree. Unrarefied data was used for the downstream analysis. The sequencing data were integrated into a single object using the “phyloseq” R package and all subsequent data analysis and plots were produced in R Studio. Alpha diversity, within sample diversity, were calculated using both richness and evenness metrics by functions estimate richness and pd in the “phyloseq” and “picante” R packages. The plots for richness estimates were generated using the “ggplot2” package in R.

Community richness was measured by observed species: total count of unique ASV's in the sample and by the nonparametric richness estimator Chao1, which accounts for the number of singletons and doubletons. Total community diversity (richness and evenness) was measured by Simpson's inverse diversity index, Shannon index, and Faith's phylogenetic diversity (PD), which uses phylogenetic distances to calculate alpha diversity.

Beta diversity, between samples diversity, was determined using phylogenetic-based Unifrac distances, which measure phylogenetic distance between samples for both unweighted (presence\absence), weighted (relative abundance), and ASV based Bray-Curtis dissimilarity matrices that accounts for both the presence/absence and abundance of unique ASV's. Beta diversity metrics were calculated with ordinate function in “phyloseq” and were visualized by non-metric multidimensional scaling (NMDS) plots using plot oridination function in “phyloseq”.

Quantitative real-time PCR was performed to determine the total bacterial load in each sequenced sample. Samples were analyzed in duplicates in a 96-well plate using a thermocycler (CFX96 Real-time system C1000 Thermocycler; BioRad Laboratories, Hercules, Calif., USA). A qPCR standard curve was generated from serially diluted Fusobacterium nucleatum ATCC 10953 genomic DNA in a range of 108 to 101 16 s copy number. Each reaction was performed in a total volume of 20 μl consisted of 2 μl of DNA or standards added to 10 μl of the master mix (TaqMan™ Fast Advanced Master Mix; Applied Biosystems, Foster City, Calif., USA). Primers set that specifically target the 16S rRNA gene were added with 900 nM final concentrations.

SEQ ID NO: 3 was the forward primer used:
5′-TCCTACGGGAGGCAGCAGT-3′.
SEQ ID NO: 4 was the reverse primer used:
5′-GGACTACCAGGGTATCTAATCCTGTT-3′.

In addition, 200 nM of TaqMan probe was used. The TaqMan probe used consisted of the fluorophore 6-carboxyfluorescein (FAM) covalently attached to the 5′-end of the oligonucleotide probe having SEQ ID NO:5 and the quencher tetramethylrhodamine (TAMRA) covalently attached to the 3′-end. Thus, the TaqMan probe had the structure, (6-FAM)-(SEQ ID NO:5)-(TAMRA). SEQ ID NO:5 is the oligonucleotide of the TaqMan probe and has the sequence:

5′-CGTATTACCGCGGCTGCTGGCAC-3′.
(Sigma Aldrich, St Louis, MO, USA).

Nuclease-free water was added to bring the total volume of the reaction to 20 μl. The negative control sample was included in the run using nuclease-free water to ensure no contamination occurred. The qPCR run consisted of the following amplification conditions: 50° C. for 2 minutes (UNG incubation); 95° C. for 20 seconds (Polymerase activation); 40 cycles of 95° C. for 3 seconds (denature) and 60° C. for 30 seconds (anneal/extend). The subgingival bacterial load was calculated (BioRad CFX software V3.1; BioRad Laboratories, Hercules, Calif., USA) using regression mode (Cq determination mode).

Because the experimental bacterial driven induction and resolution of gingivitis led to an inverted-U shape distribution of bacterial biomass (total bacterial 16S rRNA gene copies) in vivo, quadratic regression models were employed to better study the temporal changes in subgingival microbial composition and load. Quadratic regression models were implemented in R using second degree polynomials and coefficient directions and p-values representing the quadratic terms are reported.

Statistical analysis of the changes in subgingival microbial composition and load longitudinally during gingivitis induction were performed using R Software (v. 3.5.1). Logarithmic transformations (base 10) were performed for the subgingival bacterial load data prior to analysis. In a similar manner to clinical data, the difference in subgingival microbial load and alpha diversity indices between test and control sides, as well as between different responder groups were determined using linear mixed-models.

Differences in microbial community composition between test and control sides, or between different responder groups over time, were evaluated by performing permutational analysis of variance (PERMANOVA) on the calculated beta diversity matrices (Bray-Curtis and UniFrac distances) using the function adonis in the “vegan” package (v. 2.5-4). Between groups multiple comparisons were performed by pairwise PERMANOVA using the function pairwise.perm.manova from the “RVAideMemoire” package with false discovery rate (FDR) adjustment.

To identify statistically significant differences among agglomerated and normalized amplicon sequence variants (ASV's) between samples and responder groups we applied both the unpaired non-parametric Kruskal Wallis one-way analysis of variance (anova) and the nonparametric Wilcoxon-Mann-Whitney test—both with a 95% confidence interval (α=0.05) with false discovery rate (FDR) adjustment—via the “rsatix” and “ggpubr” R packages using centered log ratio (CLR) transformed data generated with the “microbiome” R package.

For statistical analyses at the genus level normalized abundances of amplicon sequence variants (ASV's) were agglomerated using a novel mean agglomeration strategy to the genus level from Day −14 to Day 35 by responder group: High, Low, and Slow (n=293 samples from 21 subjects). This mean agglomeration approach using the ASV abundances aims to offer better resolution of agglomerated abundance data at various taxonomic levels by taking into account the number of unique ASV's at that respective taxonomic level in an attempt to reduce signals that may result in an over estimation taxonomy in a sample, individual, and or group.

### Results

Control Sites are not Static and Vary by Inflammatory Responder Type

At the time of inclusion (Day −14) all study participants received a professional cleaning which aimed to normalize the oral health status across individuals. At baseline (Day 0), study subjects refrained from brushing on the test site of their mouth which was protected by a personalized plastic stint, while the control site maintained regular oral hygiene with fluoride containing toothpaste for a period of 21 Days. Plaque index (PI) (FIGS. 1A and 1B) remained rather stable among control sites over the induction period (Day 0-21). No clinical inflammation, represented by gingival index (GI) and bleeding on probing (BOP) (FIGS. 1C-1F), was observed on the control sites among these generally healthy individuals over the induction period. However, changes were observed on the control site in both gingival crevicular fluid (GCF) volume (FIGS. 1G and 1H) and bacterial load (FIGS. 11 and 1J)—although at a lower magnitude compared to test sites. Bacterial load, which represents the number of 16S rRNA gene copies within a sample, increased among all Inflammatory Responder Types (IRTs) despite a stable PI. While microbial diversity and host mediator changes have been previously reported for the test sites (Bamashmous et al., 2021 supra) these observations warranted further investigation of microbial diversity and host mediator changes within these distant control sites.

Changes in Subgingival Plaque Diversity are Observed in Healthy Control Sites and Vary by Inflammatory Responder Types

For each Inflammatory Responder Type (IRT), microbial diversity was assessed for the test and control sites. Alpha diversity analysis measured by observed amplicon sequence variants (ASVs) and Shannon indices (FIG. 2A) illustrated changes on the control and test side among IRTs. Bray-Curtis dissimilarity index was also assessed for each IRT with respect to the baseline (Day 0) and highlights a detectable increase in dissimilarity over the induction period among all IRTs test and control sites (FIGS. 2B and 2C). Beta diversity was compared using a principal coordinate analysis of Weighted Unifrac distances (FIG. 2D). The test sides showed clear shifts in beta diversity between 0 and 21 Days. On the control side, High-IRTs and Slow-IRTs displayed a similar shift, although more gradual than the test side, while Low-IRTs controls showed a relatively stable community.

Microbiome Compositions Shift within Healthy Control Sites in a Responder Dependent Manner

Subgingival plaque samples were examined at the phylum and genus levels and provided insight into the shifts in microbial diversity. Relative abundance at the phylum level was assessed by Inflammatory Responder Type (IRT) with their respective controls. The top six major phyla were assigned using the number of amplicon sequence variants (ASVs) in a phylum and relative abundance as a measure of predominance within the samples. The mean relative abundance for these phyla is highlighted in FIG. 3A. Several trends were apparent. There were distinct shifts by phyla, specifically an increase in gram-negative associated Bacteroidetes and a decrease in gram-positive associated Firmicutes. There were differences between the test and control sides as well as between the three IRTs. As seen by the Actinobacteria results, the control shifts were not just versions of the test side; rather, Actinobacteria showed increased relative abundance over the induction phase (Day 0-21) in the controls but reduced levels on the test side for the same period. The changes on the control side also appeared unlikely to be the normal maturation of the subgingival biofilm community after a deep cleaning, as some of the shifts were distinct between the IRTs.

Examining the data at the genus level, a z-scored heatmap of relative abundance was stratified by Gram stain designation (positive and negative) as well as by members of the candidate phyla radiation (CPR) (FIG. 3B). During maturation of the community on the test side a number of genera were enriched resulting in induced inflammation. Many of these genera were also enriched with the control sites, specifically: Selenomonas, Aggregatibacter, Porphyromonas, Treponema, Tannerella, Alloprevotella, Prevotella, and genera of the Saccharibacteria. However, most of these enriched members fell within the Firmicutes and Bacteroidetes phyla warranting further investigation.

The dynamics of the Firmicutes and Bacteroidetes phyla were observed within test and control sites over the induction phase for each IRT (FIG. 3C). Firmicutes decreased across all IRT test and control sites while Bacteroidetes were simultaneously increasing across all IRT test and control sites over the Induction period (FIG. 3C). This suggested that an inverse Firmicutes/Bacteroidetes Ratio (iFBR) could be implemented as an index for assessing subgingival plaque dysbiosis, with increased Bacteroidetes associated with disease and increased Firmicutes associated with homeostatic health. We plotted the iFBR for test and controls site within each IRT (FIGS. 4A and 4B). The shift from a Firmicutes dominated microbiome to Bacteroidetes occurred in the test sites by Day 4 of the Induction phase for both High- and Low-IRTs. However, the test site shift among Slow-IRTs was delayed until Day 7, characteristic of this responder phenotype (Bamashmous et al., 2021). The iFBR also shifted in control sites yet following the shifts in the test sites. The shift was delayed in the control sites until Day 7 for the High-IRTs and Day 14 for the Low- and Slow-IRTs. However, the iFBR changes seen in the control sites over the Induction phase were only statistically significant for the High- and Slow-IRTs.

Amplicon Sequence Variants are Detected Contralaterally Between Test and Control Sites

Amplicon sequence variants (ASVs) represent strain level diversity for 16S rDNA gene sequencing data (21). A total of 3,509 ASVs from enriched genera identified on the test sites during induced inflammation were examined for contralateral detection at the strain level, including: Selenomonas (19 Species, 433 ASVs), Aggregatibacter (7 Species, 155 ASVs), Porphyromonas (10 Species, 414 ASVs), Treponema (23 Species, 233 ASVs), Tannerella (3 Species, 118 ASVs), Alloprevotella (7 Species, 285 ASVs), Prevotella (38 Species, 1602 ASVs), and Saccharibacteria (8 Species, 269 ASVs) (FIG. 5D). Using a presence/absence heatmap of the 3,509 ASVs of enriched genera, 29 ASVs (0.83%) were contralaterally detected among High-IRTs with 6/6 subjects having contralaterally detected ASVs (FIG. 5A). 15 ASVs (0.43%) were detected contralaterally amongst 3/6 Low-IRT subjects (FIG. 5B). 89 ASVs (2.5%) were contralaterally detected in 8/9 Subjects among Slow-IRTs (FIG. 5C). While all the IRTs show contralateral ASV detection, High- and Slow-IRTs seemed to have higher rates in comparison to the Low-IRT. This is consistent with the higher levels observed in High- and Slow-IRTs for PI and Bacterial Load (FIGS. 1A and 1I) as well as previous characterization of the different clinical inflammatory response types (Bamashmous et al., 2021).

Maturing Plaque in Test Sites Induces Host Mediator Changes in Control Sites which Precede a Shift in the Control Site Microbiome

Having established that both the microbiome and host mediators change within control sites across Inflammatory Responder Types (IRTs), we sought to investigate if a temporal relationship between these changes could be resolved between test and control sites among the different IRTs. In order to capture when the largest shift in host mediators occurred within respective IRT control sites, we converted our host mediator data over the Induction phase (Day 0-21) into a Log Fold-Change (Log FC) value compared to Baseline (Day 0). We then plotted a z-scored heatmap of this Log FC (FIGS. 6A-6C). This revealed a distinct shift within the High-IRT by Day 4 and Slow-IRT by Day 7, including key pro-inflammatory markers TNF-a, IL-6, and IL-8 (FIGS. 6A-6F). Notably, this temporally corresponds to the shift in the two major phyla within test sites among the High- and Slow-IRT (FIGS. 6G-6I and FIGS. 4A and 4B). A temporal relationship between test and control sites among the Low-IRT was less apparent. A graphical illustration of the magnitude and timing of these changes in both subgingival community composition and host mediators among these contralateral test and control sites within respective IRTs are presented in FIGS. 6J-6K. Together, this data indicates a systemic effect in the oral cavity with a subclinical effect on the hosts mediators and microbiome within healthy sites that likely results from locally induced inflammation occurring elsewhere in the mouth. As with the microbiome and host response in the test sites, this contralateral effect appeared to vary by the different Inflammatory Responder Types identified by experimental gingivitis.

1. A method of identifying an individual as being a slow gingivitis responder or a high gingivitis responder comprising the steps of:
a) obtaining a pre-gingivitis-derived subgingival plaque sample from the individual;
b) analyzing bacterial composition of the pre-gingivitis-derived subgingival plaque sample to determine the baseline iFBR;
c) obtaining an early distant healthy-derived subgingival plaque sample from the individual;
d) analyzing bacterial composition of the early distant healthy-derived subgingival plaque sample to determine the early iFBR;
e) obtaining a late distant healthy-derived subgingival plaque sample from the individual;
f) analyzing bacterial composition of the late distant healthy-derived subgingival plaque sample to determine the late iFBR;
c) comparing the baseline iFBR, the early iFBR and the late iFBR, wherein if the early iFBR is significantly unchanged compared to the baseline iFBR and the late iFBR is significantly changed compared to the baseline iFBR and early iFBR, the individual is a slow gingivitis responder and if the early iFBR is significantly changed compared to the baseline iFBR and the late iFBR is significantly changed compared to the baseline iFBR, the individual is a high gingivitis responder.

2. A method of treating an individual who has been identified as having gingivitis comprising the steps of:
a) identifying the individual as being a slow gingivitis responder or a high gingivitis responder by the method of claim 1;
b) if the individual is identified as a slow gingivitis responder, applying to the individual's oral cavity one or more oral care compositions comprising one or more ingredients having antimicrobial activity and free of additional ingredients that have anti-inflammatory activity; if the individual is identified as a high gingivitis responder, applying to the individual's oral cavity one or more oral care compositions comprising one or more ingredients having antimicrobial activity and one or more ingredients having anti-inflammatory activity.

3. The method of claim 2, wherein the one or more ingredients having antimicrobial activity is selected from the group consisting of: arginine, zinc phosphate, zinc oxide, zinc citrate, triclosan, digluconate, thymol, menthol, eucalyptol, methyl salicylate, saline, antibiotics and fluoride.

4. The method of claim 2, wherein the one or more ingredients having anti-inflammatory activity is selected from the group consisting of: chlorhexidine, DHA and vitamin D.

5. The method of claim 2, wherein the one or more oral care compositions is selected from the group consisting of: a tooth paste, an oral rinse and a mouthwash.


SEQUENCE LISTING (5 sequences)

SEQ ID NO: 1 - DNA, 50 bp, synthetic construct
  misc_feature 1..50: note = Synthetic sequence
  variation 42: note = w = a, c, t, or g
  source 1..50: mol_type = other DNA
tcgtcggcagcgtcagatgtgtataagagacagcctacgggnggcwgcag

SEQ ID NO: 2 - DNA, 54 bp, synthetic construct
  source 1..54: mol_type = other DNA
  misc_feature 1..54: note = Synthetic Sequence
tctcgtgggctcggagatgtgtataagagacaggactachvgggtatctaatcc

SEQ ID NO: 3 - DNA, 19 bp, synthetic construct
  misc_feature 1..19: note = Synthetic Sequence
  source 1..19: mol_type = other DNA
tcctacgggaggcagcagt

SEQ ID NO: 4 - DNA, 26 bp, synthetic construct
  misc_feature 1..26: note = Synthetic sequence
  source 1..26: mol_type = other DNA
ggactaccagggtatctaatcctgtt

SEQ ID NO: 5 - DNA, 23 bp, synthetic construct
  misc_feature 1..23: note = Synthetic sequence
  source 1..23: mol_type = other DNA
cgtattaccgcggctgctggcac